(19) United States
(12) Patent Application Publication
(10) Pub. No.: US 2023/0071214 A1
(43) Pub. Date: Mar. 9, 2023

(54) MULTI-FUNCTION WEARABLE MONITORING SYSTEM WITH SLEEP DISORDER WARNING

(71) Applicant: West Affum Holdings Corp, Grand Cayman, KY (US)

(72) Inventors: David P. Finch, Bothell, WA (US); Joseph Sullivan, Kirkland, WA (US); Kenneth Cowan, Kirkland, WA (US); Korine Haynes, Woodinville, WA (US); Regina New, Henrico, VA (US); Laura Gustavson, Redmond, WA (US); Phillip Foshee, Jr., Woodinville, WA (US)

(21) Appl. No.: 17/902,508

(22) Filed: Sep. 2, 2022

Related U.S. Application Data
(60) Provisional application No. 63/240,418, filed on Sep. 3, 2021.

(51) Int. Cl. A61B 5/00; A61B 5/0205

(52) U.S. Cl. CPC A61B 5/4818; A61B 5/0205; A61B 5/746; A61B 5/4809; A61B 5/6823; A61B 2562/0219

(57) ABSTRACT

A wearable medical monitoring system is provided that includes a support structure worn by a patient, which also housing electrodes and sleep sensors for generating health data. The health data includes ECG data, respiration data, and sleep data, which are used to determine cardiac conditions and sleep disorders. Sleep disorder indexes are formulated with various combinations of sleep factors from the health data of the patient, correlating in time with particular periods of sleep by the patient. In comparing the sleep disorder indexes with respective sleep disorder indicators, potential sleep disorders may be discerned by the monitoring system. Sleep information about the sleep disorders and warnings may be transmitted by the monitoring system to health support entities.

### FIELD OF THE INVENTION

The present disclosure generally relates to use of a wearable article to monitor health factors including indications of sleep disorders.

### CROSS REFERENCES TO RELATED APPLICATIONS

This application claims the benefit of the filing date of U.S. Provisional Pat. Application No. 63/240418, filed Sep. 3, 2021, the disclosure of which is hereby incorporated herein by reference for all purposes.

### BACKGROUND

Various wearable devices can gather health related data from a person wearing the device as the person goes about day-to-day activities. Such wearables may provide data to gain a picture of specific health aspects of the person.

Some simple wearables, for instance smartwatches, can detect basic heart function. Medical-grade monitoring devices generally provide more comprehensive and reliable detection for particular health conditions, such as cardiac conditions. Medical devices used in cardiac monitoring may have electrocardiogram (ECG) electrodes that detect electrical impulses of the heart when the heart beats. Electricity detected by electrodes is translated into wavy graph lines that are recorded. Certain medical-grade monitoring devices provide critical health information that may require immediate attention, for example, lifesaving alerts of cardiac conditions, and some devices also provide treatment on the fly.

Cardiac conditions may be accompanied by other related health conditions. For example, particular sleep disorders, such as sleep apnea, can be present in patients that also have serious cardiac conditions. Sleep disorders such as sleep apnea have been associated with coronary artery disease, risk of sudden cardiac arrest, high blood pressure, abnormal heart rhythms, etc. Sleep apnea is characterized by absence of breathing during sleep. Hypopnea and respiratory effort related arousal (RERA) are other sleep disorders characterized by depressed breathing during sleep.

At times, sleep disorders can be unrecognized by some patients and medical practitioners. For example, a patient may not consider sleep disruptions as a potentially significant indications of a sleep disorder and may not inform a healthcare provider of the symptoms. As a result, the patient may not undergo testing to diagnose the sleep disorder.

### SUMMARY

A wearable medical monitoring system for health monitoring of a patient is provided to detect potential sleep disorders while monitoring for cardiac conditions. The present monitoring system acquires, assesses, and correlates various health data to determine health indications including cardiac conditions and potential sleep disorders. The multi-function monitoring system gathers a variety of health data, such as ECG data, respiration data, sleep data, patient position and movement, audio, time, etc. The monitoring system correlates the health data for a given patient with sleep periods and formulates indexes for particular sleep disorders for the patient to determine if the patient may be experiencing a sleep disorder.

The wearable medical monitoring system comprises a support structure configured for long term wear on a patient and long term acquisition of medical data including ECG and respiration data. The medical data is used for primary detection of cardiac conditions and secondary detection of sleep disorders. For example, the support structure may be configured to be worn long term on a torso of the patient. The support structure houses of various components used in the health disorders identification processes described herein.

Electrodes coupled to the support structure are positioned on the patient when the support structure is in use to detect electrical signals for generating electrocardiogram (ECG) data. One or more sleep sensors, such as an accelerometer that detects the patient motion and/or torso position data, are housed by the support structure to produce sleep signals. The monitoring system further comprises a respiration detector to generate respiration data and one or more sleep detectors to generate sleep data from the sleep signals. At least one processor is configured to use logic to perform operations that include determining a cardiac condition based, at least in part, on the ECG data. A plurality of known processes may be employed to determine the cardiac condition from the ECG data. The operations further include determining at least one respiratory disturbance during a sleep period based, at least in part, on the respiration data. The processor determines a potential sleep disorder by comparing a sleep disorder index based on sleep factors, with a sleep disorder indicator. The sleep factors are associate with two or more health data selected from a group consisting of: the respiration data, the ECG data, and the sleep data. In some implementations, the operations also include causing a communication component to transmit a warning of the potential sleep disorder to a patient support device, such as a device used by a health support entity.

In some implementations, the sleep data may comprise time of day data obtained by a clock and used to determine if the time of day is within a predefined sleep period and a predefined awake period. In various implementations, the respiration detector may receive respiratory impedance signals from alternating current (AC) signals or direct current (DC) signals at the electrodes to generate respiratory data.

In some implementations, the potential sleep disorder determined by the monitoring system may include sleep apnea. Sleep factors for a sleep apnea index may include an indication of time that the patient is asleep during the sleep period according to the sleep data. Sleep factors for apnea may also include an indication that a first subperiod of airflow is absent during the sleep period according to the respiration data. Further sleep factors for apnea may include an indication of a change in heart rate at a threshold rate corresponding with the first subperiod of airflow absence according to the ECG data. A sleep apnea determination may also be based on a torso position of the patient during the sleep period as sleep data in which the sleep factors includes an indication of a flat or angled supine position of the patient according to the sleep data.

In some implementations, the potential sleep disorder determined by the monitoring system includes hypopnea. Sleep factors for a hypopnea index may include an indication of a time that the patient is asleep during a sleep period according to the sleep data. Sleep factors for hypopnea may also include a finding that the respiration data meets a hypopnea threshold level of airflow reduction in a subperiod during the sleep period. Additional sleep factors may include one or more of a finding that oxygenation data meets a hypopnea threshold level of blood oxygen desaturation in the subperiod, and an indication of a change in heart rate corresponding with the respiration data according to the ECG data.

A hypopnea determination may also be based on an average magnitude of respiratory complexes of the respiration data acquired during the subperiod and such subperiod may be a sliding window that gets restarted upon a detection of patient motion during the sleep period. Useful for hypopnea detection, the one or more sleep sensors may comprise an accelerometer that detects the patient motion.

In some implementations, the potential sleep disorder determined by the monitoring system includes respiratory effort related arousal (RERA). Sleep factors for a RERA index may include an indication of a sleep period in which the patient is asleep during a first portion of the sleep period and awake during a second portion of the sleep period according to the sleep data. Further sleep factors for RERA may include a finding that the respiration data meets a RERA threshold level of airflow reduction during a subperiod of time during the sleep period.

A RERA determination may also be based on an average magnitude of respiratory complexes of the respiration data acquired during the subperiod. Such subperiod may be a sliding window that is restarted upon a detection of patient motion during the sleep period.

In various implementations, a method may be provided to monitor health of the patient with a wearable article of the monitoring system. Such method employs a support structure configured for long term wear on the patient, for example on a torso of the patient. The support structure includes electrodes positioned to detect electrical signals to generate electrocardiogram (ECG) data and one or more sleep sensors to generate sleep signals. Also provided are a respiration detector to generate respiration data and a sleep detector to acquire sleep data from the sleep signals.

The method includes determining a cardiac condition based, at least in part, on the ECG data, using various known techniques to evaluate cardiac conditions. Further to the method, at least one respiratory disturbance during a sleep period is determined based, at least in part, on the respiration data. The method includes determining a potential sleep disorder by comparing a sleep disorder index based on sleep factors, with a sleep disorder indicator. Such sleep factors may be associate with two or more health data selected from a group consisting of: the respiration data, the ECG data, and the sleep data. In some instances, the method may also include causing a communication component to transmit a warning of the potential sleep disorder to a communication device of a patient support user. In some implementations, an accelerometer may be employed by the method to acquire sleep data that includes patient motion data and/or torso orientation data.

In some implementations, the method is used to determine sleep apnea by sleep factors that may include an indication of a time that the patient is asleep during a sleep period according to the sleep data. Apnea sleep factors may also include an indication of a first subperiod of airflow absence during the sleep period according to the respiration data. Further sleep factors for apnea may include an indication of a change in heart rate at a threshold rate corresponding with the first subperiod of airflow absence according to the ECG data.

In some implementations, the method is used to determine hypopnea, by sleep factors that may include an indication of a time that the patient is asleep during a sleep period according to the sleep data. The sleep factors for hypopnea may also include a finding that the respiration data meets a hypopnea threshold level of airflow reduction in a second subperiod during the sleep period. Additional hypopnea sleep factors may include one or more of: a finding that oxygenation data meets a hypopnea threshold level of blood oxygen desaturation in the second subperiod, an indication of a change in heart rate corresponding with the respiration data according to the ECG data, and combinations thereof.

In some implementations, the method is used to determine RERA by sleep factors that include an indication of a sleep period in which the patient is asleep during a first portion of the sleep period and awake during a second portion of the sleep period according to the sleep data. The RERA sleep factors may include a finding that the respiration data meets a RERA threshold level of airflow reduction during a third subperiod of time during the sleep period.

### BRIEF DESCRIPTION OF THE DRAWINGS

Various implementations in accordance with the present disclosure will be described with reference to the drawings.

FIGS. 1A and 1B are diagrams of an example wearable medical monitoring system used by patients at sleep, in which FIG. 1A shows a front side and FIG. 1B shows a back side, in accordance with some implementations.

FIGS. 2A and 2B are block diagrams of example functional components of the wearable medical monitoring system, in which FIG. 2A shows components without a defibrillator feature and FIG. 2B shows the defibrillator feature that may be included with the monitoring system, in accordance with some implementations.

FIG. 3 is a schematic diagram of the example wearable medical monitoring system with a support structure, main unit, and other components, in accordance with some implementations.

FIGS. 4A, 4B, and 4C are diagrams of the example wearable monitoring system having various electrode arrangements, in which FIG. 4A is an example support structure with sensing electrodes, FIG. 4B is an example support structure with sensing electrodes and separate treatment electrodes, and FIG. 4 is an example support structure with treatment electrodes, in accordance with some implementations.

FIGS. 5A and 5B are diagrams of various types of support structure, in which FIG. 5A is an example band type support structure and FIG. 5B is an example patch type support structure, in accordance with some implementations.

FIG. 6 is a flowchart of an example method for monitoring for cardiac conditions and sleep disorders of a patient, in accordance with some implementations.

FIG. 7 is example respiration data in the form of an impedance pneumography waveform, in accordance with some implementations.

FIG. 8 is a diagram of example waveforms for health data during a sleep period indicating potential sleep apnea of a patient, in accordance with some implementations.

FIG. 9 are example waveforms for respiration data indicating sleep apnea, hypopnea, and RERA, in accordance with some implementations.

### DETAILED DESCRIPTION

In the following description, various implementations will be described. For purposes of explanation, specific configurations and details are set forth in order to provide a thorough understanding of the implementations. However, it will also be apparent to one skilled in the art that the implementations may be practiced without the specific details. Well-known features may be omitted or simplified without obscuring the implementations described. The description of the wearable medical monitoring system provides a framework which can be tailored to individual systems built around the wearable medical monitoring system. Elements may be described in terms of “basic functionality” or varying degrees of functionality.

The wearable medical monitoring system (also referred to as the “monitoring system”) includes a support structure that houses electrodes and sleep sensors, among other components for generating health data, such as ECG data, respiration data, and sleep data. The heath data are used by the monitoring system to determine cardiac conditions and sleep disorders. Sleep disorder indexes are formulated with various combinations of sleep factors applied to the health data of the patient, which correlate with particular periods of sleep by the patient. In comparing the sleep disorder indexes with sleep disorder indicators, potential sleep disorders may be discerned by the monitoring system.

A sleep disorder indicator may be a predefined quantitative and/or qualitative indication that a typical patient is likely to present with a particular sleep disorder. For example, a sleep disorder indicator may be a numerical representation derived from values assigned to each of a plurality of sleep factors associated with a particular sleep disorder, which indicate that the particular sleep factor is likely present for a given patient. For example, sleep apnea factors may include indication, e.g., factor value, that airflow is absent during a period of time and an indication, e.g., factor value, of specific change of heart rate during that period of airflow absence.

The sleep disorder indicator may be predetermined by using each factor value in a formula for the particular sleep disorder. The formula may include adding each factor value, taking a mean or average of the factor threshold, or other calculations. In some implementations, the collection of sleep factors indicate occurrence of a particular sleep disorder event and the formula for the sleep disorder indicator further includes a number of events that occur over a period of time during a sleep period (or sleep subperiod).

In some implementations, certain factor values may be weighted to effect the resulting sleep disorder indicator more significantly than other factor values. In some implementations, certain patient characteristics may influence a weight of a factor value. For example, patients with particular known genetic dispositions, age, gender, medical history, emotional patient experiences, etc., may provide for some sleep factors being weighted more heavily than other sleep factors. In this manner, a sleep disorder may be associated with a plurality of sleep disorder indicators that are defined for combinations of patient characteristics. The wearable medical monitoring system applies the appropriate sleep disorder indicator associated with the patient characteristics known for the particular patient being assessed.

A sleep disorder index may be formulated by encoding sleep factors with a same formula used to define the sleep disorder indicator, for a particular patient being evaluated and by using sleep data obtained by the wearable medical monitoring system. For example, the sleep disorder index may be formulated by adding, taking a mean value, or average value of factor values obtained by the particular patient. The sleep disorder index may be formulated by applying the same weights as employed for the sleep disorder indicator for a patient with patient characteristics which match or substantially match (e.g., within an acceptable range) those patient characteristics used in defining the sleep disorder indicator. In some implementations, the sleep disorder index includes a number of events that is found to occur for the patient over a time period of a sleep period (or subperiod of time). By encoding with a same formula, a direct comparison may be made between the sleep disorder index of the patient with the predefined sleep disorder indicator.

The wearable medical monitoring system detects potential sleep disorders in ambulator patients that are also being assessed by the monitoring system for cardiac conditions. The monitoring system may serve as a proactive detector and provide information, as well a warning, of potentially un-diagnosed sleep disorders. While the wearable medical monitoring system monitors for cardiac conditions as a primary function, the monitoring system also detects and determines signs of the one or more sleep disorders, such as sleep apnea, hypopnea, and RERA, as a secondary function. Other sleep disorders may also be detected by the monitoring system according to the processes described herein. Through the sleep information and early warning provided by the monitoring system, clinicians may follow up with further testing and/or treatment, if warranted.

The wearable medical monitoring system employs a wearable article and is configured for continuous long term wear by a patient, of at least fourteen (14) days. Some implementations of the monitoring system may be worn for a few months. The term, “continuous” is understood to include daytime use, which may be from many hours of the day and fulltime at night, to full daytime hours and full nighttime use. In some implementations, the monitoring system may be worn without stop except for temporary daytime removal during brief activities that may expose the monitoring system to potentially adverse conditions, such as water contact, e.g., bathing or swimming, cleaning of the support structure, etc. Thus, “continuous use” is intended to include such brief periods of non-use.

Some implementations may have waterproof features, such as a waterproof or water resistant housing of components and/or a support structure. In such waterproof or water resistant monitoring implementations, the monitoring system may not need to be removed when exposed to water or moist environments.

The wearable medical monitoring system includes various sensors and/or transducers, such as electrodes, to gather heath data of the patient. The term, “health data” refers to data that is generated from signals sensed, retrieved, observed, determined, and/or converted from signal source components, e.g., sensors or transducers. The health data represents a health parameter(s) of the patient or the environment of the patient, including time-related data, which may be relevant to the health of the patient.

Signal source components of the monitoring system may be configured to sense the health parameters and render an input response to a processor and/or storage. In some implementations, the input may be quantitative, such as values of a sensed parameter. In other implementations the input may be qualitative, such as informing whether or not a threshold is crossed, and so on. The term “meeting” a threshold as used in this description means a value being equal to or exceeding the threshold. In some implementations, a sensor can be construed more broadly, as encompassing more than one individual sensor.

The monitoring system includes a wearable support structure ECG data, respiration data and sleep data acquisition. In its basic form, the wearable medical monitoring system is an ECG monitoring product with at least two ECG electrodes and a device, circuitry, and/or unit to collect data for determining patient respiration and sleep characteristics of the patient.

In some implementations, the ECG data acquisition is similar to the ECG data acquisition described in U.S. Pat. No. 9,757,581, the contents of which are incorporated herein by reference. In other implementations, other types of ECG data acquisition can be used. For example, ECG data acquisition may be conducted as described in U.S. Pat. Publication No. 2017/0056682, filed Feb. 24, 2015 and in U.S. Pat. No. 8,024,037 the contents of which are incorporated herein by reference.

In various implementations, the wearable medical monitoring system may include one or more of the following features.

a) One or more devices and/or algorithm(s) to determine whether the patient was sleeping in relation to acquired electro-cardiac (ECG) and respiration data.

b) One or more devices and/or algorithm(s) to determine patient respiration over time that uses acquired patient respiration data.

c) One or more devices and/or algorithm(s) to estimate respiratory disturbances indicative of a sleep disorder including at least one of sleep apnea, hypopnea and RERA.

d) One or more devices and/or algorithm(s) to determine a sleep disorder index based, at least in part, on an average number of respiratory disturbances experienced by the patient in a defined period of time, such as an hour.

e) One or more devices and/or algorithm(s) to provide warning information of a sleep disorder index to a medical provider, e.g., clinician, for a patient as an indication of a possible sleep disorder.

Additional features and combinations of features are possible.

The wearable medical monitoring system offers benefits of monitoring a patient in a natural environment and long term monitoring. Advantages are attained over traditional polysomnography (sleep study) that is typically performed under controlled conditions of a medical facility for a short period of time, such as a night. Furthermore, the wearable medical monitoring system is configured for long term wear rather than typical short-term monitoring that may be used with other at-home sleep test systems. The present wearable medical monitoring system can collect and analyze sleep information of the patient throughout the duration of a long term wear. Such long term monitoring eliminates experimental artifacts and anomalies associated with single-day or acute sleep tests with unfamiliar equipment. Instead, the sleep patterns of the patient stabilize as the patient learns how to sleep with a long term wearable device. A clearer picture of any inherent sleep abnormalities can be obtained. Other benefits of the wearable medical monitoring system will be apparent from the further description of the system and methods, as described below.

For illustration purposes, in an example of a use case of the wearable medical monitoring system, a representative patient seeks treatment from a medical provider for cardiac episodes that he experiences. The representative patient disregards or fails to notice signs of a potential sleep disorder, such as grogginess and brain fog. The representative patient neglects to provide information that would indicate a sleep disorder to his medical provider. He dismisses sleep issues as related to daily stressors and the idea of taking time away from his busy schedule to engage in a sleep study did not initially interest the representative patient.

The medical provider in this example, prescribes the representative patient with the wearable medical monitoring system to wear continuously for fourteen days. The wearable medical monitoring system is less invasive than an implantable device, such as an implantable impedance respiration sensor, implantable cardio converter defibrillators that may implement apnea detection software, and cardiac resynchronization therapy. The present monitoring system can also monitor for arrythmias that may not occur in the short term.

The wearable medical monitoring system also includes potential sleep disorder detection. The representative patient wears the wearable medical monitoring system during his day-to-day routines as well as through the night. Tracking of normal day routine enables the representative patient to be more comfortable, and the resulting data to be more reflective of typical sleep of the representative patient. The wearable medical monitoring system is also accessible to the representative patient to monitor sleep, as no extra attention is required by the representative patient using the system for cardiac monitoring. The long term wearing of the monitoring system provides for a greater corpus of sleep data as well as daytime data and baseline data than isolated sleep studies.

The representative patient finds the torso-fitted monitoring system for continuous wear comfortable without needing to remember to put it on at night, which may occur with other recreational-use health monitors, such as health features in smartwatches and activity trackers. The medical provider appreciates the comprehensive information received from the wearable medical monitoring system including torso related health data, such as body position during sleep and esophagus functioning and status.

Although potential sleep disorders were not on the radar of the representative patient or the medical provider prior to use of the wearable medical monitoring system in this particular scenario, the monitoring system acquired various health data that indicated the representative patient may be experiencing sleep apnea. Collected health data includes ECG data, respiratory disturbance data, sleep data including sleeping body position, oxygenation levels, sleep periods, etc. The gathering of such data and transmission to the medical provider, worked in the background and appeared seamless to the representative patient. There was less risk of the acquired sleep data being negatively affected by the representative patient purposefully skewing the data, such as sleeping in a particular manner or body position to achieve a particular sleep study result, or perform other actions that may artificially impact the data.

A warning of potential sleep apnea was determined by the wearable medical monitoring system. A warning notification and a report of the gathered data was automatically pushed to the medical provider. The medical provider provided in depth assessment to diagnose the sleep problem and provide treatment to the representative patient.

In some implementations, the monitoring system may be considered an early warning device for sleep disorders, as the monitoring system may provide information to alert a medical support person of early stages of sleep disorders, for example, prior to the patient presenting with characteristics that are noticeably disruptive to the patient.

The wearable medical monitoring system is not limited to the described use case. As can be recognized by the description to herein, there are numerous other situations in which the wearable medical monitoring system may be employed, with various sensors and/or transducers to gather a variety of health data and detect numerous type of sleep disorders.

By comparison to the present wearable medical monitoring system, other types of cardiac monitors may incorporate invasive implants, e.g., an implantable defibrillator to achieve long term monitoring. Some wearable cardiac monitors, such as Holter devices, are intended for short term use and is not configured for long term wear and monitoring of a patient. For example, the present wearable medical monitoring system becomes a normal part of the patient life for a period of 14 days or more, and thus reducing risk of experimental artifact associated with Holter recordings done over a shorter time period.

Other low detection, recreational wearables, e.g., smartwatches, may also include ECG technology. Such wearables often contact electrodes to a limb of a person (“appendage type wearable”). Attachment of the device to a limb can lead to errors due to limb movement, slippage, damage to the device, cold extremities causing artifacts, etc. The position on the body of a person using an appendage-type wearable may also affect interaction of the device with the skin and the resultant signals can vary in quality.

Recreational type wearables can be susceptible to environmental elements that may damage the devices. Surface finish can alter the contact mechanisms, and some coatings can impede the flow of the EKG signal. Such recreational wearables can lack the sensitivity and accuracy to detect many cardiac issues compared to the medical grade, wearable medical monitoring system. Recreational wearables may not be capable of acquiring comprehensive data for more complete sleep information. The present wearable medical monitoring system addresses these drawbacks presented by other wearables by providing a secure wearing device with comprehensive data gathering and analysis capabilities.

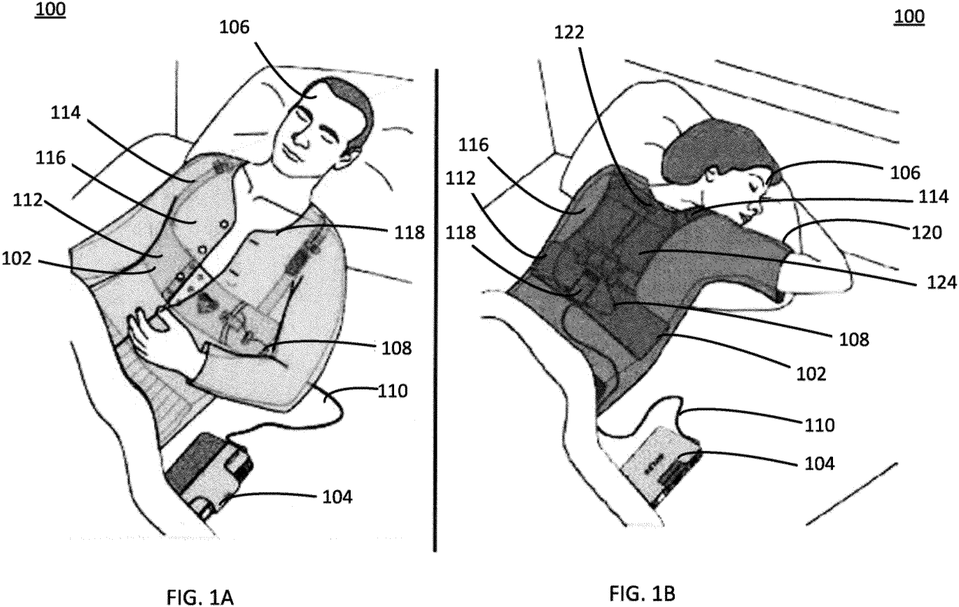

FIGS. 1A and 1B show a garment type wearable article of a wearable medical monitoring system 100 employing a support structure 102 worn on the torso of example patients 106. Torso-fitted support structures may reduce risk of damage that can occur with monitoring device attached to a limb of a person, such as the wrist or ankle, especially for patients with an active lifestyle. The monitoring system may also include a main unit 104 and various circuitry 108 and/or cables 110 for communication between components. The wearable medical monitoring system 100 is shown to illustrate concepts about the monitoring system deployed on a patient while asleep during a sleeping period. FIGS. 1A and 1B are provided to illustrate concepts about the support structure 102, and these figures are not to be construed as limiting how the support structure 102 is implemented, or how the support structure is worn.

The patient 106 may also be referred to as a person, user, and/or wearer. Patient 106 is ambulatory, which means that while wearing the monitoring system 100, the patient 106, when awake, is capable of walking, ride in a vehicle, and so on. In other words, the patient 106 is not necessarily bed-ridden.

While the patient 106 may be considered to be also a “user” of the monitoring system 100, the term “user” as used in this description, is not exclusive to the patient 106. For instance, a user of the monitoring system 100 may also be a medical provider such as a clinician, doctor, nurse, emergency medical technician (EMT), or other similarly tasked and/or empowered individual or group of individuals. In some cases, a user may even be a bystander. The particular context of these and other related terms within this description should be interpreted accordingly.

The support structure 102 can be implemented in many different ways to provide a structural platform for particular components of the monitoring system, such as signal source components that generate signals representing various health parameters associated with the patient and a hub 118 (shown in FIG. 1B) that may collect and/or process signals from the signal source components. Components may be detachably held by various pockets, receptacles, inserts, straps, removeable attachment mechanisms, e, g., hook and loop, etc.

The support structure may be a single structural element, or a combination of multiple structural elements. The example support structure 102 shown in FIGS. 1A and 1B is a vest garment style support structure which is based on fabric material. The support structure 102 includes a main body portion 112 fitted around a torso 116 of the patient 106 and two shoulder straps 114 with one shoulder strap over each shoulder of the patient 106 from the front side of the patient to the back side of the patient. The support structure may also be implemented with a single shoulder strap, for example, that may wrap around the neck, or around one shoulder at an angle, or as a full vest rather than having shoulder straps. A back body portion 124 shown in FIG. 1B may couple with the shoulder straps on the back side of the patient. The main body portion 112 may be installed to fit snug around the torso with the shoulder straps 114 holding the main body in place onto the torso 116 of the patient 106. In this manner, the support structure 102 may encircle the patient without a need for adhesives to attach the support structure onto the patient. The main body portion 112 may be enclosed around the torso with fasteners, such as snaps, buttons, hook and loop, clasps, clamps, buckles, catchers, ties, etc., or with flexible fabric or elastic to allow stretch for slippage onto the torso. Other enclosures are possible.

The depictions in FIGS. 1A and 1B are not to be construed as limiting how the support structure 102 is implemented or worn. The support structure 102 can be implemented in many different ways to engage with at least a portion of the torso of the patient and provide for long term continuous monitoring of the patient. For example, it can be implemented in a single element or a combination of multiple elements, which may be coupled together. In some implementations, support structure 102 could include a vest, a half-vest, or other type of garment that engages with at least a portion of the torso. In some implementations, support structure 102 could include a harness, one or more belts or straps, etc. that fit on a torso or other accessible parts of the patient. The support structure 102 can also be worn around hips, over the shoulder, around appendages, etc. In implementations, such items can be worn similarly to analogous articles of clothing. Such items can be worn parallel to or underneath other articles of clothing, such as a pajama shirt 118, 120.

In some implementations, the support structure can be worn by being attached to the patient’s body by adhesive material, for example as shown and described in U.S. Pat. No. 8,024,037. The support structure 102 may also be implemented as described for the support structure of U.S. Pat. Application No. US2017/0056682, which is incorporated herein by reference. In such implementations, the person skilled in the art will recognize that additional components of the monitoring system can be in a housing of a support structure instead of being attached externally to the support structure, for example as described in the US2017/0056682 document.

The support structure 102 may include a sleep position detector 122 (shown in FIG. 1B), such as a 3-D accelerometer, that recognizes the sleep position to be used in a collection of sleep data. The sleep position detector 122 may be positioned on the back body portion 124 of the support structure to fit on the upper back torso of the patient.

The sleep position detector may detect that the patient 106 in FIG. 1A is sleeping in a position on the patient’s back with the head raised from the torso at about a 45 degree angle at the location of the sleep position detector 122 with respect to a standing position assumed to be perpendicular to the floor (for example zero degrees is a straight standing position and lying flat is considered ninety degrees). Other points of reference to determine angle body position are possible. The sleep position detector 122 may detect that the patient 106 in FIG. 1A is sleeping in a position on the stomach with the head of the patient raised at the torso at about a 60 degree angle at the location of the sleep position detector 122.

In implementations, the support structure 102 can include one or more containers or housings, which can be waterproof. A person skilled in the art will recognize that in some implementations, additional components of the wearable medical monitoring system can be in a housing of the support structure 10 instead of attached externally to the support structure.

The support structure 102 may be made of a variety of fabrics. In some implementations, material of the support structure may also include conductive fabric for transportation of electric signal and/or power to and/or from components of the wearable medical monitoring system.

The support structure may be removed from the patient, for example, prior to the patient engaging in brief activities and replaced onto the patient afterwards. In some implementations, the support structure can be detachably secured to the patient by adhesive material. Various components, including electronic components including sensors, batteries, electrodes, and cables, may be detached from the support structure, for example, to wash the support structure, repair, or replace the support structure.

Signal source components include sensors, transducers, electronics, etc. to detect various health parameters for generating health data used in sleep factors to formulate a sleep disorder index for the patient. Health parameters may include patient physiological parameters (heart rate, breathing characteristics, blood oxygenation, body temperature), patient state parameters (body position and orientation), system parameters, environmental parameters, and so on. In some implementations, a patient may provide consent to the wearable medical monitoring system detecting storing, and transmitting particular health parameters, such as audio, associated with the patient.

Physiological parameters of the patient detected by the monitoring system may include ECG, electrical impedance, DC current signals, respirational characteristics, blood oxygen level, blood flow, blood pressure, blood perfusion, pulsatile change in light transmission or reflection properties of perfused tissue, heart sounds, heart wall motion, breathing sounds, pulse, etc.

Patient state parameters detected by the monitoring system may be used to determine sleep status of the patient. Patient state parameters may include recorded aspects of patient, such as motion, posture, whether they have spoken recently, what they said, and so on, plus optionally the history of these parameters.

Environmental parameters detected by the monitoring system can include ambient temperature and pressure. Moreover, a humidity sensor may provide information as to whether it is likely raining. Presumed patient location could also be considered an environmental parameter.

In some implementations, system parameters of monitoring system can also be detected, including system time characteristics, such as date and time of day. Other system parameters may include a system identification, battery status, reports of self-testing, records of data entered, episodes, and intervention stored in memory of the monitoring system, and so on.

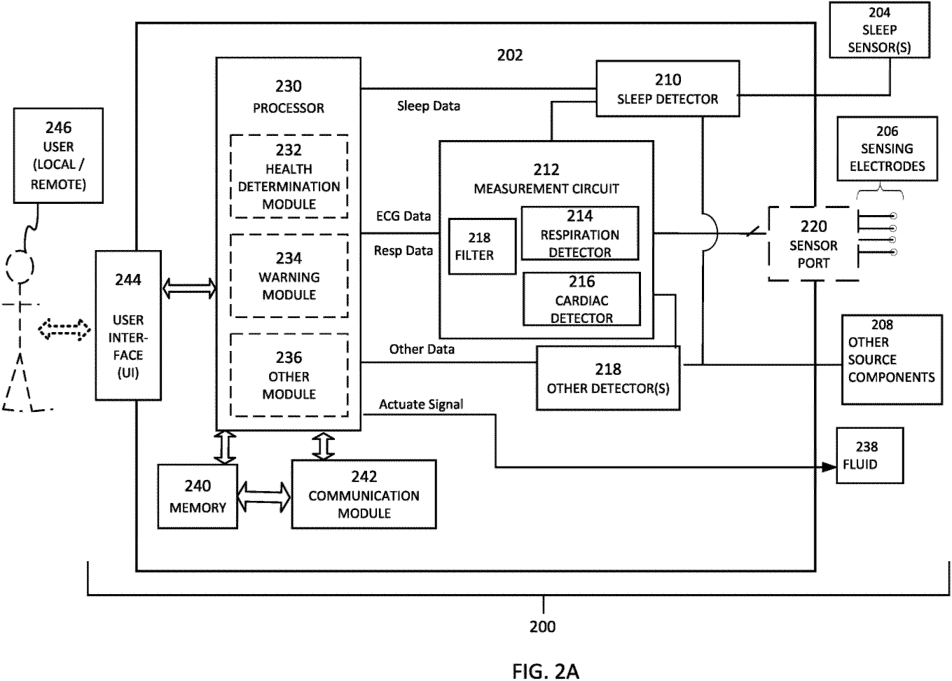

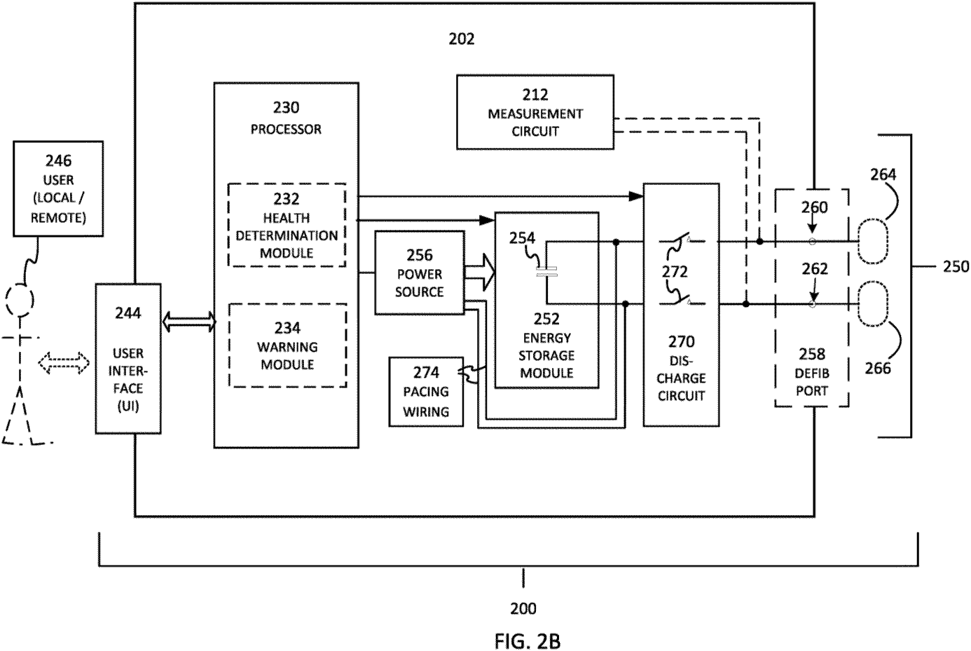

In some implementations, parameters detected by the monitoring system may include a trend that can be determined based on stored historical data that the by the processor of the monitoring system compares with currently acquired data. Parameters trends that can particularly useful for a cardiac rehabilitation program, for example, include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO₂ or CO₂; f) respiratory function, respiration rate, etc.; g) motion, level of activity; and so on. Once a trend is detected, it can be stored and/or reported via a communication link, along with, perhaps, a warning. From the report, a user monitoring the progress of patient will know about a condition that is either not improving or deteriorating. Source components and health parameters are described in further detail below, for example with regard to FIG. 2A.

The main unit 104 of the wearable medical monitoring system is configured to perform various processes for operations of the monitoring system, such as receiving health data, storing various data and information, implementing treatment, performing operations on health data, and determining potential and/or actual medical disorders, e.g., sleep disorders, cardiac disorders, respiratory disorders, etc. The main unit 104 may also provide information, power, and/or instructions to the hub 118 of the support structure, or directly or indirectly via the hub, to signal source components of the monitoring system.

The main unit 104 may further serve to output information to a computing device of a health support entity and/or the patient. The main unit 104 may be in communication with one or more components of the support structure 102, for example, via wired cables or wireless communication such as Bluetooth or ZigBee connections, and other communication mechanisms. In some implementations, the main unit 104 may communicate and interact with the hub 118 on the support structure 102, which may transfer signals from the signal source components to the main unit 104. More details of functions of the hub 118 and main unit 104 are described below, for example with regard to FIGS. 2A and 2B.

During a sleeping period of the patient, the main unit 104 may be placed at a proximal distance away from the patient, such as on a bed or table. When the patient is awake and mobile, the main unit remains in communication with the components, such as the hub 118. During awake periods, the main unit may be transported by the patient by various modes, such as in a pack or purse carried by the patient, on a belt, by a strap over the shoulder, or additionally by further adapting the support structure 102, and so on.

As shown in FIGS. 2A and 2B, the wearable medical monitoring system 200 employs a variety of sensors, transducers, and/ other source components to generate and/or transfer health signals associated with the patient to unit 202 of the monitoring system 200. The unit 202 is intended to represent one or more units that perform functions on health signals or health data. Functions of the unit(s) can include one or more of (1) collect signals representing various health parameters relevant to the health of a patient from signal source components (2) generate health data associated with the signals, (3) determine potential or actual medical disorders, and (4) output information about the medical disorders. The unit 202 may also perform additional functions and combinations of these functions (1)-(4).

In some implementations, the functions of unit 202 may be entirely performed by a single unit, such as the main unit 104 in FIG. 1 of the monitoring system. In other implementations, particular functions represented by unit 202 may be shared between two or more units, such as a hub 118 in FIG. 1 and the main unit 104. For example, the hub may be housed in the support structure 102 in FIG. 1 and may be in direct communication with signal source components, such as sleep sensors 204, sensing electrodes 206, and other source components 208, to collect heath parameter signals generated by such signal source components. The hub 118 may also transfer the signals or health data associated with the signals to the main unit 104. The main unit 104 may process data, store data, use data for various determinations, including sleep disorder indexes, respiratory disturbances, and cardiac disorders, and/or output information/data to a user 246. In other implementations, a main unit 104 is in direct communication with signal source components without use of a hub to collect health signals. Thus, the unit 202 depicted in FIGS. 2A and 2B is intended to include one or more units, which can include a hub and main unit, which individually perform any combination of the functions described.

In some implementations, at least one unit that performs some of the functions represented by unit 202 may be located at a remote place away from the patient and the support structure. In such configurations, the remote unit may communicate with a hub or signal source components via a wireless communication. A unit that is remote may include a cloud computing device, e.g., server, to process data retrieved by components of the monitoring system 200. For example, in some implementations, health data may be stored in memory on a local unit and uploaded to a remote unit for further processing, e.g., via a night time connection with a remote unit.

The unit 202 may communicate with a variety of signal source components, such as sleep sensors 204, sensing electrodes 206, and other source components 208. Signal source components include sensors and/or transducers, to collect health signals associated with health parameters relevant to the monitoring system 200 determining a health disorder, such as cardiac and sleep characteristics of the patient. Health parameters may include any combination of patient physiological parameters, patient state parameters, system parameters, and environmental parameters. Other types of signal source components are possible, including clocks to track time and date. Signals from the various source components feed into detectors for processing and generating of associated health data.

Sleep sensor 204 detects sleep related health parameters and produces sleep signals accordingly. The sleep signals are fed to a sleep detector 210 of unit 202, which generates sleep data from the signals. Sensing electrodes 206 produce heart activity related signals, such as ECG signals and respiratory signals, which are fed into a corresponding cardiac detector 216 and/or respiratory detector 214 of a measuring circuit 212 to generate ECG and/or respiration data.

A health determination module 232 of processor 230 applies health data received from the detectors to combinations of health factors and formulates health disorder indexes that characterize the health conditions of the patient. For example, combinations of sleep factors are applied as rules or criteria in determining a sleep disorder index and similarly in defining a sleep disorder indicator. The health disorder index is a summary of how well or to what degree health data of the patient satisfies the combination of health disorder factors. In some implications, a health disorder index may be a rating of the how well the health data satisfies the factors. The health determination module 232 compares the formulated health disorder index for the patient to a health disorder indicator that is designated for the health disorder and determines whether the index is within a threshold amount of the indicator sufficient to suggest the patient is potentially afflicted with the health disorder.

Other signal source components 208 may also produce signals, which are transferred to other detector(s) 218 to generate other data that may be used by other module 236 of the processor 230, for example to maintain operation of the wearable medical monitoring system 200.

In some implementations, a signal source component may serve multiple roles in producing signals used to generate different types of health data. For example, a sensing electrode may provide both ECG signals and respiratory signals. At times, a particular type of signal may be used to interpret different health impacts of a parameter, such as signals from an accelerometer may be used in determining cardiac conditions by cardiac detector 216, for example if a motion signal indicates a patient exercises at a time that correlates with usual cardiac activity. The accelerometer signals may also be used by sleep detector 210 to determine a sleeping body position or orientation associated with particular sleep disorders, e.g., obstructive sleep apnea. The accelerometer may also be used to monitor breathing patterns of the patient as an indicator of sleep status. For example, the accelerometer may provide signals associated with chest motion, e.g., rise of chest indicating breathing in and chest lowered indicating breathing out. Thus, the support structure may house an accelerometer that produces different types of signals for different types of health data, or may house more than one accelerometer dedicated to produce signals for particular types of health data.

Sensing electrodes 206 may also produce different types of signals for ECG interpretations and for respiration assessments. Sensing electrodes 206 be one or more transducers configured to acquire electrical signals indicative of heart activity, such as ECG signals and respiratory signals, e.g., impedance signals from variating AC current and DC current signals. Unit 202 may optionally have at least one sensor port 219 in unit 202, which can be also known as an ECG port when used for ECG signals. Sensor port 219 is adapted for plugging in sensing electrodes 206, which, in some implementations, may also be known as ECG electrodes and ECG leads. An ECG signal may be, for example, a 12-lead signal, or a signal from a different number of leads. Sensing electrodes 206 can be attached to the inside of the support structure 102 shown in FIG. 1, for making good electrical contact with the patient. The ECG of the patient can be sensed as a voltage difference between sensing electrodes 206.

In some implementations, unit 202 also includes a measurement circuit 212 to receive one or more electrical physiological signals of the patient from sensor port 220. Physiological input to the cardiac detector 216 of the measurement circuit 212 may reflect an ECG measurement.

Electrical signals in the form of impedance signals may be fed as input to the respiratory detector 214 of the measurement circuit 212 to determine impedance respiratory data. Impedance can be sensed between electrodes 206 and/or the connections of sensor port 220. In implementations, respiratory detector 214 may be configured to render impedance respiratory data of the patient based on received impedance AC signals that can be rendered as a modulation to a carrier signal, as a stream of values, and so on. The measurement circuit 212 can render or generate health data about the received signals. The health data rendered by measurement circuit 212 is provided as output and this health data can be input relative to a subsequent device or functionality, such as processor 230.

The processor 230 may determine the correlation between impedance change and the volume of respirated air. For example, an increase in impedance may be detected due to the patient breathing in and increasing the gas volume of the chest in relation to the fluid volume, resulting in a decrease in conductivity. To further cause an increase in impedance, breathing in also results in a lengthening the conductance paths because of expansion, further increasing impedance. The impedance respiratory data allows for measuring respiration of the patient. Sensing the impedance can also be useful for detecting other details, such as whether the sensing electrodes 206 are not making good electrical contact with the patient’s body.

In some implementations, unit 202 may also include one or more filters, such as a filter 218 of the measurement circuit 212 configured to receive signals rendered by respiration detector 214 and/or ECG detector 216, such as the ECG signal, impedance signal, and so on. Filter 218 can be configured to derive a filtered impedance signal from a rendered impedance signal. The filtered impedance signal may correspond to the rendered impedance signal, with at least a portion of the rendered impedance signal changed. For example, a filtered impedance signal may be derived by removing variations from rendered impedance signal variations that have a frequency greater than a threshold frequency, such as 10 Hz. Filtered impedance signal may also be derived by removing from rendered impedance signal variations that repeat over a period of time, such as at least 30 seconds. Some example methods of impedance pneumography that may be applied in some implementations, are described in Amit K. Gupta, “Respiration Rate Measurement Based on Impedance Pneumography, Application Report” SBAA181, Feb. 2011, pp. 1-11, Texas Instruments Incorporated, Dallas, Texas.

In some implementations the sensing electrodes 206 may be used to measure low-frequency changes in a DC level of a signal. For example, in some implementations, a small DC current may be injected into at least two sensing electrode 206, and the DC voltage of that electrode relative to a reference electrode may be indicative of the electrode resistance. Pressure on the electrodes in contact with the skin may change with each breath, which may affect the resistance of the electrode. Changes at the interface between the person’s skin and the electrode may be detected as small changes in resistance of the electrode when the small DC signal is injected. Some example methods of DC signal detection to generate respiration data, which may be applied in some implementations, are described in U.S. Pat. Publication No. 2021/0100457, filed Oct. 1, 2020, the contents of which are incorporated by reference.

Sleep sensors 204 of the wearable medical monitoring system 200 provide signals, used in generating sleep data which characterize sleep and/or awake states of the patient. In some implementations, sleep sensors may include a motion sensor, such as an accelerometer to sense body position, motion events, or orientation, etc. for sleep data used in sleep factors by the health determination module 232 identifying sleep disorders. A motion event can be indicated, for example by a patient change in motion from a baseline motion or rest, etc.

The sleep detector 210 may accept signals of an accelerometer to generate sleep data that indicates when the patient is asleep by detection of body motion, orientation, or position. Determination of sleep periods may also correlate this sleep data from the accelerometer with other types of data from other signal source components. For example, the patient may be found to be asleep when the patient is detected as lying with little or no motion at an overlapping time that ECG data indicates slightly lower heart rate by ECG signals of the sensing electrodes, and further correlates in time with respiration data that indicates slightly lower respiration by impedance or DC current respiration signals of the sensing electrodes.

In some implementation, a 3-axis accelerometer may be employed to detect various aspects of sleep and awake, such as determine sleeping position of the patient, times that the patient has woken up during a sleep period and times the patient has gone back to sleep. The accelerometer sleep sensor may be positioned at or near the torso of the patient to detect orientation of the torso, for example the accelerometer may be positioned on the support structure in the upper back area of the patient. The 3-axis accelerometer may detect that a patient is lying on the back, left side, right side, or stomach. Body sleeping position and orientation may be used in a sleep factor to determine potential sleep disorders, such as obstructive sleep apnea.

In some implementations, the accelerometer may be readily adapted for use with the present teachings by those skilled in the art, as discussed in terms of a 3-axis accelerometer more fully in U.S. Pat. No. 11,083,906, filed Jan. 5, 2018, the contents of which are incorporated into this disclosure by reference. In some implementations, some features of the accelerometer may be used as described in U.S. Pat.No. 11,344,718, the contents of which are incorporated into this disclosure by reference. Other accelerometer devices and techniques may be employed to determine breathing characteristics, such as described in U.S. Pat. No. 10,159,421.

The sleep sensor may be fixed on the support structure at a position to sense the orientation of a particular body part compared to a baseline orientation. For example, an accelerometer may be placed on the sleep structure proximal to the upper mid-back of the patient to sense the torso relative to gravity. In some implementations, the accelerometer may be placed in other places of the support structure on or proximal to the torso such as on the front of patient, the lower back of the patient, or proximal to the neck of the patient, for example in a position that detects an angle of the esophagus of the patient.

The health determination module 232 may correlate torso position with highs and lows of other data, such as ECG data, respiration data, etc. For example, the health determination module 232 may find a numerical sleep disorder index of 1 when the patient is determined to be lying on the stomach and the sleep disorder index may be a 15 when the patient is lying on the back, which is also found to correlate with a greater number of respiration events detected according to the respiration data. In this example, a higher numerical index is indicative of high potential of a sleep disorder as further indicated by comparison with a sleep disorder indicator.

The sleep sensor may also include other sensors useful by health determination module 232 in determining sleep characteristics, such as a global positioning system (GPS), a clock to provide time of day and date, etc. In some implementations, body position sensors may be positioned on a substrate that the patient lies on, as an external signal source, rather than fixed to the support structure, for example, as described in U.S. Publication No. 2021/0022621, filed Jul. 27, 2020, the contents of which are incorporated into this disclosure by reference. In such implementations, sleep signals from the external signal source are wired or wirelessly transferred to the unit 202 to convert to sleep data for use by the monitoring system.

In some implementations, sleep data may be applied to negative sleep disorder factors for logic of the health determination module 232 to rule out a sleep disorder or sleep period of the patient. For example, although a clock may reflect a typical sleep period of a particular patient, an accelerometer and/or GPS may indicate the patient is active and walking rather than sleeping. In this manner, correlating health data from a variety of signal source components can decrease a risk of false positive or false negative findings.

Examples of other signal source components 208 may include sensors or transducers such as a perfusion sensor, a pulse oximeter, a device for detecting blood flow (e.g. a Doppler device), a sensor for detecting blood pressure (e.g. a cuff), an optical sensor, illumination detectors and sensors working together with light sources for detecting color change in tissue, a device that can detect heart wall movement, a sound sensor, a device with a microphone, a Saturation of Peripheral Oxygen (SpO₂) sensor, a GPS for example used to determine a sudden cardiac arrest or other cardiac conditions, and so on. Signals from the other signal source components 208 may be inputted into the cardiac detector 216, respiratory detector 214, sleep detector 210, and/or other detector(s) 218. For example, signals representing rate or type of activity of a patient in motion by an accelerometer may assist in the health determination module 232 to determine cardiac conditions in which a heart may be strained.

Certain signal source components 208 can help detect a pulse of the patient as pulse rate sensors. Pulse detection is also taught by way of example in U.S. Pat. No. 8,135,462, filed on Apr. 17, 2008, which is hereby incorporated by reference in its entirety. In addition, a person skilled in the art may implement other ways of performing pulse detection. For example, a signal source sensor for a heart sound may include a microphone, etc.

Other signal source components 208 may include environmental sensors such as sensors to sense environmental conditions of the patient. In some implementations, sensing of temperature, pressure, moisture, may provide environmental signals that the sleep detector may interpret in determining sleep, cardiac distress, or other patient conditions. For example, prolonged ambient temperature may be a factor in determining that a patient is asleep and that a sleep period has commenced.

The health data is processed by the processor 230 of the wearable medical monitoring system 200. The processor 230 may be implemented in a number of ways. Such ways include, by way of example and not limitation, digital and/or analog processors such as microprocessors and Digital Signal Processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 may include, or have access to, a non-transitory storage medium, such as a memory 240. Such a memory 240 can have a non-volatile component for storage of machine-readable and machine-executable instructions. A set of such instructions can also be called a program or logic. The instructions, which may also referred to as “software,” generally provide functionality by performing methods as may be disclosed herein or understood by one skilled in the art in view of the disclosed implementations. In some implementations, and as a matter of convention used herein, instances of the software may be referred to as a “module” and by other similar terms. Generally, a module includes a set of the instructions so as to offer or fulfill a particular functionality. Implementations of modules and the functionality delivered are not limited by the implementations described in this document.

Processor 230 can be considered to have a number of modules. One such module can be a health determination module 232 to determine a variety of actual or potential health disorders including those health disorders discussed above such as cardiac conditions and sleep disorders. For example, the health determination module 232 may implement logic to determine a cardiac condition based, at least in part, on the ECG data and a potential sleep disorder, based at least in part, on the sleep data. The health determination module 232 combines various sleep factors into a sleep disorder index that may implicate a particular sleep disorder. The sleep factors are used as rules to which the sleep data is applied in formulating a sleep disorder index for the patient.

In an example, the health determination module 232 can implement logic to determine a Ventricular Fibrillation (VF). The ECG data from measurement circuit 212, which can be available as physiological inputs, data, or other signals, may be used by the health determination module 232 to determine whether the patient is experiencing VF. Determining of a VF can provide information of a possible resulting sudden cardiac arrest. The health determination module 232 can also include logic to determine a Ventricular Tachycardia (VT), and other cardiac conditions.

The health determination module 232 can also implement logic to determine a respiratory disturbance using the respiration data. For example, airflow reduction levels may be sensed and represented in the respiration data.

The health determination module 232 applies particular health data, e.g., sleep data, to health disorder factors, e.g., sleep factors, that together may indicate potential health problems, e.g., sleep disorders. The health determination module 232 determines a health disorder index based on finding that the health disorder factors are satisfied or unsatisfied by the health data. The health determination module 232 may compare the health disorder index with a health disorder indicator to determine a potential disorder of the patient. For example, a sleep disorder index is based on sleep factors that includes a combination of health data, such as the respiration data, the ECG data, and the sleep data.

In some implementations, when the health determination module 232 finds an occurrence of a respiratory disturbance of the patient during a sleep period, the health determination module 232 may be triggered to explore particular sleep disorders that may be associated with the respiratory disturbance. In this manner, sleep disorder indexes for one or more sleep disorders are generated upon finding of a respiratory disturbance.

The processor 230 may also include a warning module 234, which generates information such as health data, and/or a warning of a determined medical condition or potential medical condition based on outputs of the health determination module 232. The warning may take a variety of formats, such a report with particular wording of the determined condition, or other signals of the determined condition such as a color or symbol in a report, audio output, a visual light, etc. The warning may be received by and outputted at a health support entity device and/or a patient device.

The processor 230 can also include additional modules, such as other module 236, to perform other functions for multiple purposes. For example, the other module 236 may operate particular source components or other devices.

The unit 202 further includes a memory 240, which can work together with processor 230. Memory 240 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 240 is thus a non-transitory storage medium. Memory 240 can include programs for processor 230, which processor 230 may be able to read and execute. More particularly, the programs can include sets of instructions in the form of code, which processor 230 may be able to execute upon reading. Executing is performed by physical manipulations of physical quantities, and may result in functions, operations, processes, actions and/or methods to be performed, and/or the processor to cause other devices or components or blocks to perform such functions, operations, processes, actions and/or methods. The programs can be operational for the inherent needs of processor 230, and can also include protocols and ways that decisions can be made by advice module 234. In addition, memory 240 can store prompts for user 246 if this user is a local rescuer.

Memory 240 may be employed to store data, such as health data. This data can include patient data, system data and environmental data, for example as learned by the various source components. The data can be stored in memory 240 before it is transmitted out of unit 202, or stored there after it is received by unit 202.

Optionally, the wearable medical monitoring system 200 may also include a fluid 238 that it can deploy automatically between the sensing electrodes 206 and skin of a patient. The fluid can be conductive, such as by including an electrolyte, for establishing a better electrical contact between the electrode and the skin. Electrically speaking, when the fluid 238 is deployed, the electrical impedance between the electrode and the skin is reduced. Mechanically speaking, the fluid may be in the form of a low-viscosity gel, so that it does not flow away from the electrode, after it has been deployed. The fluid can be used for both defibrillation electrodes 264, 268 shown in FIG. 2B, and for sensing electrodes 206.

The fluid 238 may be initially stored in a fluid reservoir, not shown in FIG. 2, which can be coupled to the support structure. In addition, a fluid deploying mechanism 274 can be provided to cause at least some of the fluid 238 to be released from the reservoir, and be deployed near one or both of the patient locations, to which the sensing electrodes 206 are configured to be attached to the patient. In some implementations, the fluid deploying mechanism 274 is activated by an actuation signal from the processor 230.

Unit 202 may also include a power source 240. To enable portability of unit 202, power source 240 typically includes a battery. Such a battery may be implemented as a battery pack, which can be rechargeable, or not. Sometimes a combination of rechargeable and non-rechargeable battery packs are used. Other implementations of power source 240 can include an AC power override, for where AC power will be available, an energy-storing capacitor, and so on. In some implementations, power source is controlled by processor 230. Appropriate components may be included to provide for charging or replacing power source 240.

Unit 202 can optionally include a communication component for establishing one or more wired or wireless communication links with other devices of health support entities, such as a remote assistance center, Emergency Medical Services (EMS), and so on. The communication component may include various hardware and/or software elements, such as a communication module 242 and/or user interface 244. The communication component may also be used to communicate with a device of the patient. For example, information regarding determinations of a cardiac condition and/or sleep disorder, including a warning of potential disorders, may be wirelessly transmitted to a device of the patient, such as a smartphone, and also to a device of the health support entity.

The communication module may also include software that enables communications of the user interface 244 over a network such as the HTTP, TCP/IP, RTP/RTSP, protocols, wireless application protocol (WAP), IEEE 902.11 protocols, and the like. In addition to and/or alternatively, other communications software and transfer protocols may also be used, for example IPX, UDP or the like. The communication network may include a local area network, a wide area network, a wireless network, an Intranet, the Internet, a private network, a public network, a switched network, or any other suitable communication network, such as for example cloud networks. The network may include many interconnected computer systems and any suitable communication links such as hardwire links, optical links, satellite or other wireless communications links such as Bluetooth, Wi-Fi, wave propagation links, or any other suitable mechanisms for communication of information. For example, the network may communicate to one or more mobile wireless devices, such as mobile phones, tablets, and the like, via a base station such as a wireless transceiver.

In some implementations, communication module 242 may transmit wirelessly, on a daily basis, patient health data such as heart rate, sleep data (raw data or as applied to sleep factors), sleep disorder indexes, respiratory rate, and other vital signs data to a server accessible over a network, such as the internet, for instance as described in U.S. Publication No. 2014/0043149, filed Aug. 6, 2013. This data can be analyzed directly by the a medical provider of the patient and can also be analyzed automatically by algorithms designed to detect a developing conditions and then notify medical personnel via text, email, phone, etc.

In addition, communication module 242 may also have the capability to contact emergency services when an episode of sudden time critical condition, such as a cardiac death, is detected or other critical illnesses are detected. Communication module 242 may also include such interconnected sub-components as may be deemed necessary by a person skilled in the art, for example an antenna, portions of a processor, supporting electronics, outlet for a telephone or a network cable, etc. In this way, data, commands, etc. can be communicated.

Unit 202 can optionally include other components. In some implementations, one or more of system components may be customized for the patient. This customization may include a number of aspects. For instance, support structure 170 can be fitted to the body size, shape, or particular characteristics, such as wheelchair bound, of the patient.

In some implementations, customization may be based on detected parameters of the patent. For example, baseline physiological parameters of patient can be measured, such as the heart rate of patient while resting, while walking, motion detector outputs while walking, etc. Such baseline physiological parameters can be used to customize the wearable medical monitoring system, in order to make its diagnoses more accurate, since bodies differ from one patient to another. Such parameters can be stored in a memory 240 of the unit 202.

User 246 may include a person and/or computing system of a person or entity. For local interaction with the wearable medical monitoring system 200, the user 246 may include the patient or a local bystander. In some implementations, the user 246 may be a remote entity, such as a computing device of a remote person or a medical server device. For example, the user 246 may be a health support entity such as a doctor, caregiver, other health care provider, health care service, dispatch, technical service, an authorized person, and so on, including combinations thereof. The user may also include a medical server device or devices such as a cloud service, serving as a repository for health data of the patient.

A user interface 244 may transmit information to the user 246 and in some implementations may receive information from the user 246. The information may be pushed to user 246 in the form of a warning, in response to detecting a potential sleep disorder, cardiac condition, or other health problems. For example, the warning may be a statement that a patient is at risk of having a particular sleep disorder and the information may provide a quantitative risk of such sleep disorder in the form of a sleep disorder index compared to a sleep disorder indicator defining a risk threshold for the sleep disorder, e.g., a risk finding of a sleep disorder index of 15 for sleep apnea compared to a sleep disorder threshold of 12, may indicate a high potential that the patient experiences sleep apnea, as the sleep disorder index is found to be greater than the sleep disorder threshold. In some implementations, the warning may include a risk symbol, such as colors, asterisks, etc., to indicate a risk level. For example, a bubble or highlighting of a color red may point to high risk, yellow for medium risk, and green for low risk of the sleep disorder.

In some implementations. Presenting information to user 246 as an indication of a possible sleep disorder is by providing periodic, such as daily, trend information over selected intervals of time (e.g., 1 week, 1 month, etc.). These trends allow a user to identify significant changes in a health disorder index that may correlate with other health disorder indexes and/or symptoms of the patient. For example, it may be found that a first index for a first health disorder, such as a cardiac condition, may trend towards a first health disorder indicator. It may further be found that another index for a second health disorder, such as sleep apnea, is also trending closer to a second health disorder indicator at a similar rate as the first index. Even though the two health conditions may not yet meet its respective health disorder indicator, correlation of the two trends may suggest to a health support entity that an onset of a cardiac condition and/or sleep disorder may be imminent unless treated.

In some implementations, trend data for a health disorder index may be presented to a health support entity that could differentiate index values for different torso orientations. This trend data may enable comparisons of index values for different patient torso orientations that may be indicative of obstructive sleep apnea, rather than other types of sleep apnea. A patient with an obstructive sleep apnea condition may show significantly higher sleep apnea index values when the patient is supine compared to when the patient is prone or laying on the side.

In some implementations for presenting health disorder information to a user, may involve trend-based presentation of separately collected patient information along with the sleep disorder index to provide a correlation and/or confidence metric for the sleep disorder index data. Examples of separately collected patient information that could be presented along with the sleep disorder index trend data may include: (i) daily sleep questionnaire “sleepiness scale” results; (ii) sleep questionnaire “sleepiness scale” results gathered on days following sleep periods with high sleep disorder index values; and (iii) Sleep quality scores determined by a separate mobile phone application.

In some implementations for presenting the sleep disorder index information to a user, continuous “episode” recordings may be collected and displayed of ECG, respiration and other available vital signs information covering a time prior to, during, and after respiratory events that produced high sleep disorder index values. In this manner, the health support entity may view the acquired information and look for typical respiratory disturbance signatures such as changes in heart rate, respiration depth and reduced blood oxygen saturation level.

The output information may include a comprehensive report of sleep disorder findings with various index and sleep factor results. The information may include the findings for each sleep factor, e.g., a period of airflow reduction by a specified amount or absence of airflow correlated with heart rate anomalies during the period during a sleep period, which comprises a determined sleep disorder index. For example, for hypopnea the information may include sleep data indicating times that the patient is asleep and awake during a defined sleep period, respiration data meeting a hypopnea threshold level of airflow reduction during a subperiod of the sleep period, oxygenation data that meets a hypopnea threshold level of blood oxygen desaturation during the sleep period, ECG data indicating a change in heart rate, or combinations thereof. It should be recognized that various other types of monitoring information and forms of output of the information, such as tables, graphs, and images, are possible.

In some implementations, particular information may be pushed to the user 246 at predesignated times or intervals. The information may also be pulled to user 246 by requests for information sent to the unit 202 by user 246 or other authorized parties, for example according to the Health Insurance Portability and Accountability Act of 1996 (“HIPAA”) or other privacy regulations, to access the information.

In some implementations, the user interface 244 may include output devices, which can be visual, audible or tactile, for communicating to a user by outputting images, sounds or vibrations. Images, sounds, vibrations, and anything that can be perceived by user 246 can also be called human-perceptible indications. Example output devices can include a light, or a screen to display what is sensed, detected and/or measured, and provide visual feedback to user 246 for their resuscitation attempts, and so on. Another output device can be a speaker, which can be configured to issue voice prompts, beeps, loud alarm sounds and/or words to warn bystanders, etc.

User interface 244 may further include input devices for receiving inputs from users. Such input devices may additionally include various controls, such as pushbuttons, keyboards, touchscreens, one or more microphones, and so on. An input device can be a cancel treatment switch, which is sometimes called an “I am alive” switch or “live man” switch. In some implementations, actuating the cancel switch can prevent an impending delivery of a shock or other treatment.

In some implementations the user may include a technical specialist to maintain or repair operations of the wearable medical monitoring system, or to input health data for use by the processor. For example, the user interface 244 may serve as a programming interface to receive predefined assessment data used in determinations by processor 230, such as previously measured baseline physiological parameters, health disorder indicators used in comparison with determined health disorder indexes, patient characteristics and history data, etc. Such a programming interface may input automatically in the monitoring system 200 the assessment data, and such data may be stored in memory 240.

Another data flow can be a communication through user interface 244 between a local user and remote person. Communication can be a voice conversation, email messaging, texting, and so on. The doctor can query the patient, the patient can ask questions of the doctor, and so on. In addition, communication can be automatically issued reminders by a medical server, and so on.

In some implementations, the monitoring system 200 can be configured to provide cardiac treatments directly to the patient, such as defibrillating the patient, in addition to monitoring health parameters. Defibrillation can be performed by defibrillate components of the monitoring system delivering an electrical charge to the body of the patient in the form of an electric shock. The electric shock can be delivered in one or more pulses.

In some implementations the wearable medical monitoring system 200 shown in FIG. 2A may be supplemented with components to provide treatment to the patient based, at least in part, on the health determination of processor 230 from the monitoring results of the patient. As shown in FIG. 2B, a defibrillation feature 250 may be included in the monitoring system 200 with components to provide automatic defibrillation. FIG. 2B illustrates the defibrillation feature that may be inclusive to (merged with) the components of the monitoring system 200 shown in FIG. 2A.

The defibrillation feature 250 of the unit 202 may include an energy storage module 252 to temporarily store electrical energy in the form of an electrical charge, when preparing it for discharge the pulse to administer a shock. Energy storage module 252 can be coupled to the support structure of the wearable medical monitoring system, for example either directly or via defibrillation electrodes 264, 265 and their leads. In implementations, energy storage module 252 can be charged from a power source 256 to a designated amount of energy, as controlled by processor 230. In typical implementations, energy storage module 252 includes a capacitor 254, which can be a single capacitor or a system of capacitors, and so on. In some implementations, energy storage module 252 may include a device that exhibits high power density, such as an ultracapacitor. Capacitor 254 can store the energy in the form of an electrical charge, for delivering to the patient.

Based on the findings of a cardiac disorder by the processor 230, the processor 230 may further determine that treatment is warranted. In some implementations, the processor 230 may determine treatment based on additional information as well, such as patient medical history data, event history data, etc. The processor 230 may activate discharge circuit 270 to deliver an appropriate shock treatment to the patient. In some implementations, when the determination is to shock, an electrical charge pulse is delivered to the patient. Delivering the electrical charge is also known as discharging. Shocking can be for defibrillation, pacing, and so on.

A defibrillation capable unit 202 may also include defibrillation port(s) 258, such as a socket in housing of the unit 202. Defibrillation port 258 includes electrical nodes 260, 262. Leads of defibrillation electrodes 264, 266 can be plugged into defibrillation port 258, so as to make electrical contact with nodes 260, 262, respectively. It is also possible that defibrillation electrodes 264, 266 are connected continuously to defibrillation port 258, instead. Either way, defibrillation port 258 can be used for guiding, via electrodes, the electrical charge that has been stored in an energy storage module 252, to the patient. The electric charge (also referred to as “pulse”) will be the shock for defibrillation, pacing, and so on.

Unit 202 moreover includes a discharge circuit 270. When the decision is to shock, processor 230 can be configured to control discharge circuit 270 to discharge through the patient the electrical charge stored in energy storage module 252. When so controlled, discharge circuit 270 can permit the energy stored in energy storage module 252 to be discharged to nodes 260, 262, and from there also to defibrillation electrodes 264, 266, so as to cause a shock to be delivered to the patient.

Discharge circuit 270 can include one or more switches 272, which may also include one or more bridges. Switches 272 can be made in a number of ways, such as by an H-bridge, cross-bar switch, or other switching mechanisms to control current flow. Discharge circuit 272 can also be controlled by a user or external computing device via user interface 244. Measuring circuit 212 can further monitor the amount of electrical current provided from the discharge circuit 272 prior to release to the patient.

When the defibrillation electrodes 264, 266 make sufficient electrical contact with the body of the patient, the unit 202 can administer, via the defibrillation electrodes 264, 265, a brief, strong electric pulse through the body. The pulse is also known as defibrillation pulse, shock, defibrillation shock, therapy, electrotherapy, therapy shock, etc. The pulse is intended to go through and restart the heart, in an effort to save the life of the patient. The defibrillation pulse can have an energy suitable for its purpose, such as at least 100 Joule (“J”), 200J, 300J, and so on.

In some implementations, defibrillation electrodes 264, 266 may be multi-functional to also provide electrical signals to measurement circuit 212 to generate ECG data and/or respiratory data as described above for FIG. 2A. In such implementations, the monitoring system 200 may include both defibrillation electrodes 264, 266 and sensing electrodes 206, or may include the multi-functional defibrillation electrodes 264, 266 without also dedicated sensing electrodes 206.

In some implementations, cardiac treatment may include providing a pacing pulses with energies similar to pacers rather than defibrillators. Pacing type treatments may be performed to address congestive heart failure when the heart is unable to pump sufficiently to maintain blood flow to meet the needs of the body. The unit 202 may implement a pacer instead of, or in addition to, a defibrillator. The wearable medical monitoring system 200 may detect when the heart rhythm of the patient starts to deteriorate and has not yet reached a state where the patient needs to be defibrillated. In such implementations, the monitoring system 200 may pace the patient first, and not have to resort to the full intervention of defibrillation. Of course, if the patient does not respond to the pacing and the heart rhythm deteriorates further, the monitoring system may then later cause one or more defibrillation shocks to be delivered.

For pacer implementations, at least some of the stored electrical charge can be caused to be discharged via at least two of the defibrillation electrodes 264, 265 through the patient, so as to deliver to the patient a pacing sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 100J, 25J, usually about 10J, and so on. In either case, the pulse has a waveform suitable for this purpose.

When the decision of the determination module 232 is to provide electrical discharge in the form of a pace, i.e., to deliver pacing pulses, the processor 230 can be configured to cause control the discharge circuit 270 to discharge through the patient at least some of the electrical charge provided by the power source 256. Since pacing requires lesser charge and/or energy than a defibrillation shock, in some implementations, pacing wiring 274 is provided from the power source 256 to the discharge circuit 270. The pacing wiring 274 is shown as two wires that bypass the energy storage module 252. As such, the energy for the pacing is provided by the power source 540 either directly via the pacing wiring 274, or through the energy storage module 252. And, in some implementations where only a pacer is provided, the energy storage module 252 may not be needed if enough pacing current can be provided from the power source 256.

In some implementations, advice is outputted through user interface 244 to convey a shock or no shock determination of the processor 230, for example via warning module 234. The shock/no shock determination can be made by executing a stored Shock Advisory Algorithm. A Shock Advisory Algorithm can make a shock/no shock determination from one or more ECG signals that are captured according to implementations, and determining whether a shock criterion is met. The determination can be made from a rhythm analysis of the captured ECG signal or otherwise.

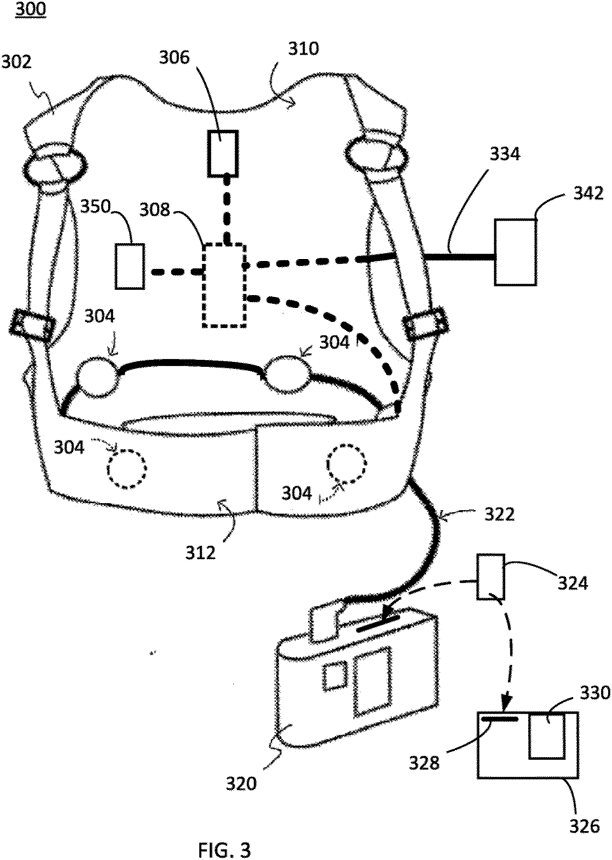

FIG. 3 depicts components of the wearable medical monitoring system 300 having a vest type garment support structure 302 housing various components, one or more external signal source components 332 that are not housed by the support structure, and a main unit 320 in communication with the support structure and that perform various functions described above with regard to FIGS. 2A and 2B.

Components held by the support structure 302 may include multiple electrodes 304 and a sleep sensor 306, such as an accelerometer. The support structure 302 may also contain a hub 308 to communicate with the various signal source components of the support structure 302, as well as communicate with one or more external signal source components 332, such as an oximeter, not housed by the support structure 302. The hub 308 may further communicate with the main unit 320 and perform some of the functions described with regard to unit 202 in FIGS. 2A and 2B described above.

Additional signal source sensors 350 that may be housed by the support structure 302 may include an audio detector such as heart-sound/phonocardiogram, patient temperature thermometer, GPS, pressure sensors, optical sensors such as photoplethysmography, etc. to produce signals used in generating health data about the patient. Such additional signal source sensors 350 may be wired or wirelessly coupled to hub 308 to provide signals produced by the additional signal source sensors 350 according to the sensors sensing of parameters of the patient.

An audio detector may be provided to produce signals to represent sounds of the body of the patient, e.g., heart sounds, breathing sounds, and/or sounds of the surrounding area of the patient. The audio detector may sense heart activity, blood flow, snoring, or the patient talking. Placement of the audio detector on the support structure provides for targeted detection. For example, a microphone may be positioned to detect frequency, volume, intensity, regularity, etc. of heart beat. The microphone may also detect frequency, volume, regularity, etc. of breaths.

Some examples of audio sensors that may be employed in some implementations are described in U.S. Pat. No. 11,058,884, the contents of which is incorporated by reference. Breath audio data may include frequency, volume, regularity, etc. of breaths. A frequency that is above a frequency threshold may indicate shortness of breath. Snoring may also be detected as sleep data to indicate the patient is asleep. Breath data may be coupled with other data to determine whether the patient is asleep, such as low ambient lighting, time of day, motion sensors, etc. Heart audio data may include frequency, volume, intensity, regularity, etc. of heart beats. Other audio sensors can be used to generate other audio data.

External signal source component 342 may be attached to other parts of the patient, for example, which may be more conducive to sensing a physiological parameter rather than at the torso. An example external signal source component 332 may include an oximeter (also referred to as an “SpO2 sensor”) engaged with a body part, such as a finger, of the patient in which blood flow is easily detected. The oximeter may detect a reduction in blood oxygen level and signals or health data from the oximeter can be time-synchronized with other health data, such as the ECG and respiration data. Reduced blood oxygen saturation level may indicate a respiratory disturbance of the patient.

Another example of an external signal source component 342 may include an environmental audio detector, such as a microphone, to detect environmental sounds, such as snoring, talking, noise in the surrounding area of the patient, etc. Environmental sounds sensed by the audio detector may provide signals relevant to the state of the patient, such as whether the patient is in a bedroom sleeping or on the go.

The hub 308 receives data from the external signal source component 342 and transfers instructions or activation signals to the external signal source component 340 via wired cable connection 334 and/or a wireless communication mechanism. In some implementations, an external signal source component 342 may transfer signals directly to main unit 320 rather than to hub 308.

Various communication mechanisms may be employed between components of the monitoring system, such as among support structure components including hub 308, electrodes 304, sleep sensor 306, and additional signal source components 350 and between external components such as main unit 320 and external signal source components 342. Such components may be attached via wires to certain other components. Other communication mechanisms between components are possible. For example, some implementations may employ a conductive material in the support structure 312, such as conductively enhanced fabric comprising the support structure, to provide for passage of electrical communication signals from component to component through the support structure.

Wireless communication mechanisms may include Bluetooth, radiofrequency, Zigbee, Wi-Fi, near field communication (NFC), infrared communication, GPS, and other wireless communication technologies. Wireless communication may employ security protocols to protect health signal, data, and information.

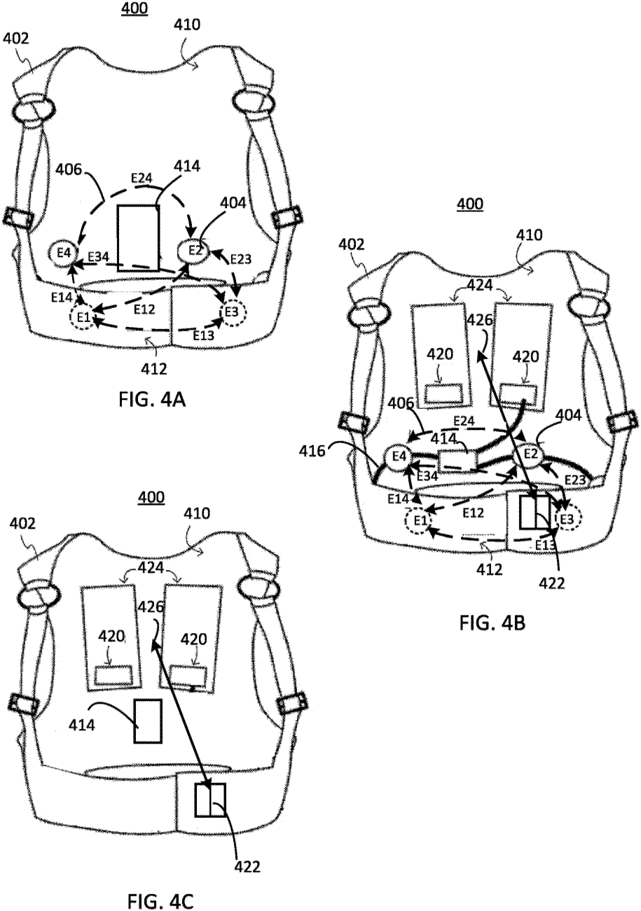

Electrodes 304 are removably fixed to an inside of the support structure 302 to make contact with the skin of the patient directly or through a conductive medium, such as an electrolyte. The electrodes 304 may be electrically coupled to main unit 320 or to the hub 308 via electrode cable 322. The electrodes 304 may be functional as both therapy and monitoring electrodes, or just monitoring electrodes without therapy functionality, as described below for FIGS. 4A-4C. Electrodes 304 may be configured to produce electrical signals for ECG data and/or respiration data, e.g., AC signals for respiratory impedance determination, or DC signals.

The electrodes 304 can be configured to be worn by the patient in a number of ways. For instance, the main unit 320 and the electrodes 304 can be coupled to the support structure 170, directly or indirectly. In other words, the support structure 302 can be configured to be worn by the patient so as to maintain the electrodes 304 on the body of the patient, while the patient is moving around, etc. The electrodes 304 can be maintained on the body attachment to the skin of the patient, simply pressed against the skin directly or through garments, etc. In some implementations the electrodes 304 are not necessarily pressed against the skin, but become biased that way upon sensing a condition that could merit intervention.

In addition, some of the components of the main unit 320 can be considered coupled to the support structure 302 directly, or indirectly via at least one of the electrodes 304. For example, main unit 320 may be directly connected to hub 308 coupled to on an exterior side 312 of the support structure. In some implementations, electrodes are positioned to contact the back and/or front of the patient by the support structure. For example, the electrodes may be configured on the support structure 302 to snuggly engage with skin over the rib cage of the patient below the breast area and above the stomach.

The main unit 320 may include a battery 324 (shown in an exploded view) that provides power to the monitoring system 300. The battery is inserted into a slot in the main unit 320 when in operation. The battery 324 is often rechargeable and a charged spare battery may be employed to swap while the battery 324 is charging.

A charging station 326 may be provided to recharge a battery of the main unit. The charging station may include a slot 328 for charging and/or a charging well 330 for wireless charging. In some implementations, the battery may be removed from the main unit and inserted into the charging station. Other mechanisms to recharge the battery are possible, such as the battery being configured to receive wireless charge. For example, the battery may include one or more coils to receive inductive charge. In other implementations, the battery may include contacts that correspond with an external power source to receive conductive charge.

In some implementations, the main unit 320 may receive signals or data from the hub 308 and perform computations to determine whether the patient experiences a health disorder, including cardiac conditions and sleep disorders. In other implementations, the main unit 320 may perform some processing of data from the hub and transmit the processed data to a remote computing device (not shown) to make determinations of heath disorders.

As shown variously in FIGS. 4A, 4B, and 4C the wearable medical monitoring system 400 may be configured for monitoring only or monitoring and treatment. These figures show a garment type support structure 402 having components for monitoring and/or treatment.

FIG. 4A illustrates an example wearable medical monitoring system 400 for monitoring only without treatment. A garment support structure 402 excludes dedicated defibrillation components that would otherwise be employed for treatment purposes. Support structure 402 includes sensing electrodes 404 (E1, E2, E3, E4).

Any pair of the four sensing electrodes 404 (E1, E2, E3, E4) defines a vector 406 (E12, E13, E14, E23, E24, E34) along which an ECG signal may be sensed and/or measured. It should be recognized that other numbers of ECG electrodes are possible such as 2 ECG electrodes for example E1 and E12, 5 electrodes, etc. Accordingly, any number of vectors may be defined by the sensing electrodes 404.

Vectors 406 (E12, E13, E14, E23, E24, E34) define channels. ECG signals may thus be sensed and/or measured from channels respectively, and in particular from the appropriate pairings of wire leads for each channel.

Support structure 402 has a back side 410 and a front side 412 that closes in front of the chest of the patient. Support structure 402 is configured to be worn by the patient so as to maintain sensing electrodes 404 on a body of the patient. Sensing electrodes 404 (E1, E2, E3, E4) are maintained on the torso of patient, and have respective wire leads. Sensing electrodes 404 (E1, E2, E3, E4) may surround the torso for sensing ECG signals, impedance, and/or DC signals.

Hub 414 on support structure 402 communicates with the sensing electrodes 404 (E1, E2, E3, E4) to send control signals and to receive sensing signals. Wires 416 connect sensing electrodes 404 (E1, E2, E3, E4) to each other and to hub 414.

FIG. 4B is an example of a monitoring system 400 with combined monitoring and treating components. The garment support structure 402 includes both ECG sensing electrodes 404 and dedicated defibrillation components including posterior defibrillator electrodes 420 and anterior defibrillator electrodes 420. A defibrillator vector 426 is formed by pairing of posterior defibrillator electrodes 420 and anterior defibrillator electrodes 420.

Support structure 402 is configured to be worn by the patient so as to maintain electrodes 404 on a body of the patient. Posterior defibrillation electrodes 420 are maintained in pockets 424 of the support structure 402. The inside of pockets 424 can be made with loose netting, so that posterior defibrillator electrodes 420 can contact the back of the patient, especially with the help of the conductive fluid that has been deployed. In addition, sensing electrodes 404 are maintained in positions that surround the patient’s torso, for sensing ECG signals and/or the impedance of the patient.

Wires 416 connect sensing electrodes 404 (E1, E2, E3, E4) to each other, to hub 414 and to defibrillator electrodes 420, 422. The sensing electrodes 404 (E1, E2, E3, E4) define vectors as described above for FIG. 4A. The description of components and features described above with regard to FIG. 4A apply to components and features labeled and not described specifically for FIG. 4B.

FIG. 4C is an example of a monitoring system 400 employing electrodes for combined functions of monitoring and treatment, in accordance with some implementations. The garment support structure 402 includes defibrillation components excluding ECG electrodes. In such implementations, defibrillation electrodes 420, 422 may also serve to capture ECG signals and/or respiratory signals across the two or more defibrillation electrodes 420, 422.

Posterior defibrillator electrodes 420 and anterior defibrillator electrodes 420 define defibrillator vector 426. The description of components and features described above with regard to FIGS. 4A and 4B apply to components and features labeled and not specifically described for FIG. 4C.

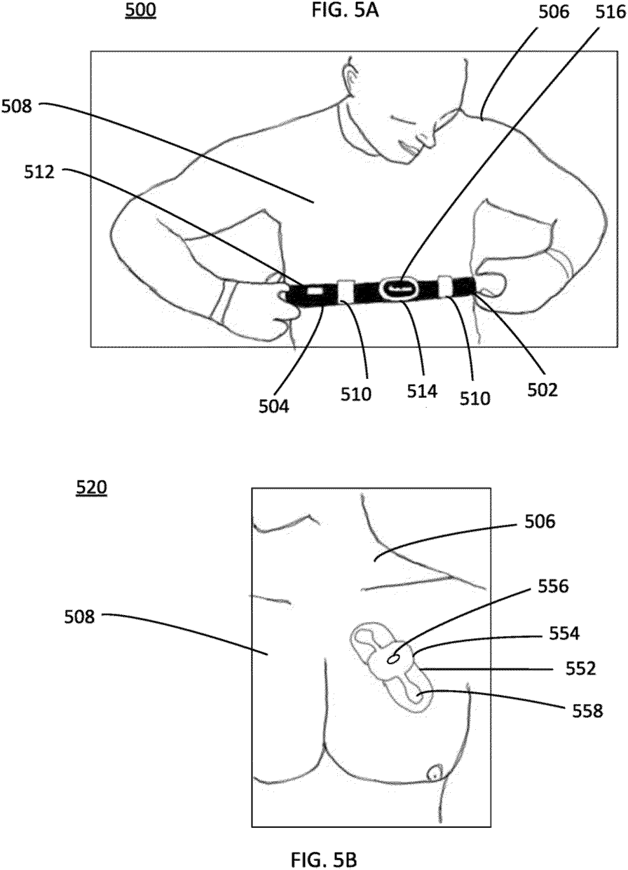

The support structure shown in FIGS. 4A-4C illustrate a vest type garment support structure. In some implementations, the support structure may take various forms or combination of forms. For example, the support structure may include one or more patches, one or more bands, and/or a torso fitted garment to house various components of the wearable medical monitoring system. FIG. 5A is an example wearable medical monitoring system 500 with a band type support structure 502 and FIG. 5B is an example wearable medical monitoring system 520 with a patch type support structure 552.

The band type support structure 502 in FIG. 5A includes a band 504 that extends around a torso 508 of a patient 506. A hub 514 may be situated on the band to communicate with system components of the wearable medical monitoring system 500. The hub 504 may be in communication with signal source components, e.g., electrodes 510 and sleep detector 512.

Two or more sensing electrodes 510 are positioned along the band. For example, two sensing electrodes 510 may be placed on the band to contact the front of the torso of the patient and two sensing electrodes may be placed on the band to contact the back of the patient (not shown). In some implementations the sensing electrodes 510 may also serve as defibrillator sensors to release an electric shock to the patient for treatment of cardiac disorders. One or more sleep detectors 512 may also be coupled to the band.

In some implementations, various system components may be configured to be external of the band and in communication with on-band components. Examples of external components may include signal source components, such as an oximeter, audio detector, temperature gauge, etc. A main unit may also be employed external of the band and may communicate with the hub 514 and/or signal sense components, e.g., sensing electrodes 510 and sleep detector 512.

The band may include a fastener 516 to removably attach the band to the body and tighten the band for a secure and close contact with the patient. In some implementations, the band may include an adhesive on an interior side to further establish a secure and close interaction with the skin.

As shown in FIG. 5B, wearable medical monitoring system 520 having patch type support structure 552 includes a housing 554, a sleep sensor 556, and two or more sensing electrodes 558 extending from the housing 554.The patch type support structure 552 is configured to engage with the patient 506 at the torso 508.

The patch type support structure 552 shown in FIG. 5B may be configured to attach to the skin via adhesives. Sensing electrodes 558 may extend from the support structure 552. ECG data acquisition may take place across the two or more sensing electrodes 558 provided on the patch. Within a housing 554 may be one or more additional sensors. One or more motion-based breath sensors such as accelerometers may be included for accelerometer data collection, for example, to detect chest motion from breathing. Other sensors such as audio sensors may also be employed with the patch type support structure 552, such as microphones, for audio data collection to identify or characterize breathing sounds and/or heart sounds.

Within the housing 554, the patch type support structure may include the unit 202 as shown in FIG. 2A. In some implementations, the patch type support structure may include a transmitter to wirelessly send signals from the sensors and electrodes to a main unit for processing, and may or may not include an onboard processor. Examples of patch devices in which, in some implementations, certain features may be incorporated in the patch-type support structure are described in U.S. Pat. No. 10,159,421 and in U.S. Pat. No. 9,615,763.

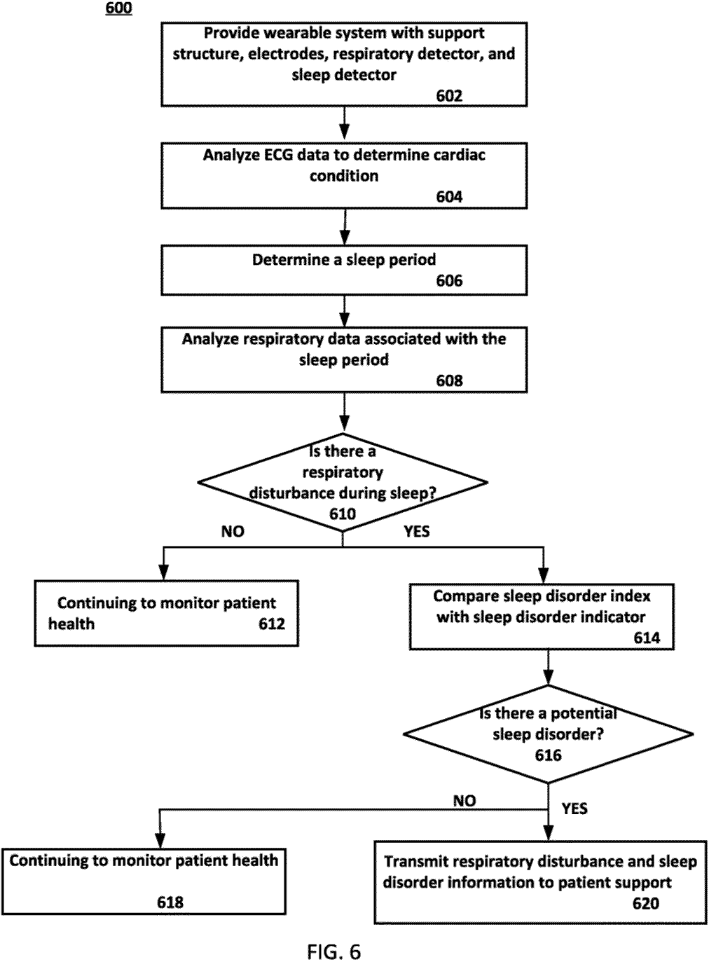

FIG. 6 shows a flowchart of an example medical monitoring process 600 of a patient. The medical monitoring process is performed by components of the wearable medical monitoring system, for example 200 of FIG. 2A. In block 602, the wearable medical monitoring system is provided. The monitoring system has a support structure with electrodes, a respiratory detector, and sleep detector. In some instances, the support structure houses a hub that includes a cardiac detector, a respiratory detector, and a sleep detector. Some or all of the process 600, or any other processes described herein, or variations and/or combinations of those processes, may be performed under the control of one or more computer systems configured with executable instructions and/or other data, and may be implemented as executable instructions executing collectively on one or more processors. For example, the process 600 may be adapted to include steps for detection of particular sleep disorders such as sleep apnea, hypopnea, and RERA, as described. The process 600 may be also adapted to include steps to detect other sleep disorders.

In block 604, ECG data are analyzed to determine a cardiac condition. The cardiac information may be provided to a computing device of a health support entity, such as a medical practitioner. The cardiac information may include an alert of a cardiac condition that the wearable medical monitoring system determines based on the analysis. Cardiac information may also include cardiac related health data including circumstances that no cardiac condition event has been determined. Such cardiac related health data may include a sampling of ECG data that does not indicate a cardiac condition. For example, the health data may include a warning that patient health data are approaching a threshold that indicates a cardiac condition event, but such a threshold has not yet been met. In some examples, the cardiac related health data may include other information associated with the patient that may be pertinent to health, such as patient activity, environmental conditions experienced by the patient, time of day and date information, etc. Known processes using health data detected by the monitoring system to determine cardiac conditions may be employed.

Cardiac information may be pushed to the health support entity on a regular basis, in response to an event such as a determined cardiac condition or a warning of an approaching cardiac condition event. The cardiac information may also be pulled on demand by the health support entity, for example, through user interface 244 in FIG. 2A.

In block 606, a sleep period for the patient is determined as a period of time during a day that the patient is asleep for at least a subperiod of time and is expected to be sleeping or attempting to sleep. The sleep period may include one or more subperiods of awake, which can be temporary, and subperiods of sleep. Thus, the patient may be asleep during an entire sleep period, or may periodically wake up during the sleep period.

In some implementations, sleep detection is provided by a time-of-day clock in the wearable medical monitoring system in combination with pre-defined sleep periods. Time-of-day sleep periods can be fixed, for example, nightly from 10 pm to 6 am, or can be customized by the patient, such as according to a work schedule of the patient.

The sleep period may also be determined by various combinations of sleep data including one or more of patient health data, patient status data, environmental data, time of day, etc. Combinations of sleep period data may reduce false positive indications of sleep periods, for example, sleep periods that occur during the day. In some implementations, a combination of sleep data that indicates a sleep period may be customizable as specific to a particular patient. In this case, the sleep period may be predefined for the patient that consistently sleeps during a specific time of day. For example, a particular patient may always go to sleep or attempt to sleep at a particular time period during the night.

In some implementations, detecting patient sleep does not need to be determined in real time. For example, accelerometer data may be acquired, stored, and analyzed in a “batch mode”. In some implementations, the wearable medical monitoring system can be selectively configured into a batch mode and/or a real time mode.

A marker that may be detected for assessing a patient sleeping may be body activity, such as lack of any significant motion, e.g., lack of significant motion in any axis of a 3-axis accelerometer, to indicate that the patient is still for a threshold period of time. Markers for sleep may also include body position in which a torso angle of the patient is horizontal (approximately 0 degrees) or within a tilt range that indicates the patient is not standing or sitting up (e.g., +/- 45 degrees). For example, in some implementations, to obtain the above metrics and/or detect sleep, one or more accelerometers can be used to detect the patient’s posture, sleep incline or angle, the patient’s angle of sitting, reclining, and/or patient movement or activity.

A marker for sleep may further include heart rate data, e.g., at rates that indicates sleep cycles, etc. In still other implementations, the sleep period may be predefined and fixed for all patients according to a particular time of day. The monitoring system may also be configured to use sensed environmental data, such as dark ambient light, as a factor in determining a sleep period.

In some implementations, sleep periods can be determined by combining two or more of the following sleep period factors that may indicate sleep independently or in various combinations, including: (i) time according to a time-of-day clock; (ii) analysis of patient accelerometer data that reflect occurrence of sleep; (iii) reduction in heart rate (average or lowest-rate sliding window) that often occurs during sleep; and (iv) sleep period indications provided by a separate, wirelessly connected mobile device app, etc. Combinations of sleep factors may facilitate improvement in sensitivity and reduction of false positive indications of sleep periods.

In block 608, analysis is performed on respiration data representing patient respiration activity during a sleep period of the patient. Analysis may include comparing the acquired respiration data to expected respiration data, such as baseline respiration data for the patient or to other typical respiration data. Discrepancies that are found by the comparison may indicate an occurrence of a respiratory disturbance event of the patient. Various respiration data may be analyzed using a variety of techniques, such as impedance pneumography and DC voltage change detection.

Monitoring systems that use impedance pneumography, may use an alternating current (AC) carrier frequency and measure AC impedance between two electrodes, e.g., sensing electrodes 206 in FIG. 2A or defibrillator electrodes 264, 266 in FIG. 2B. Changes in impedance are detected as a marker for patient breathes. Examples of impedance pneumography that may be employed are described in U.S. Pat. No. 11,052,241, and in Gupta, Respiration Rate Measurement Based on Impedance Pneumography, Texas Instruments, Application Report SBAA181, February 2011.

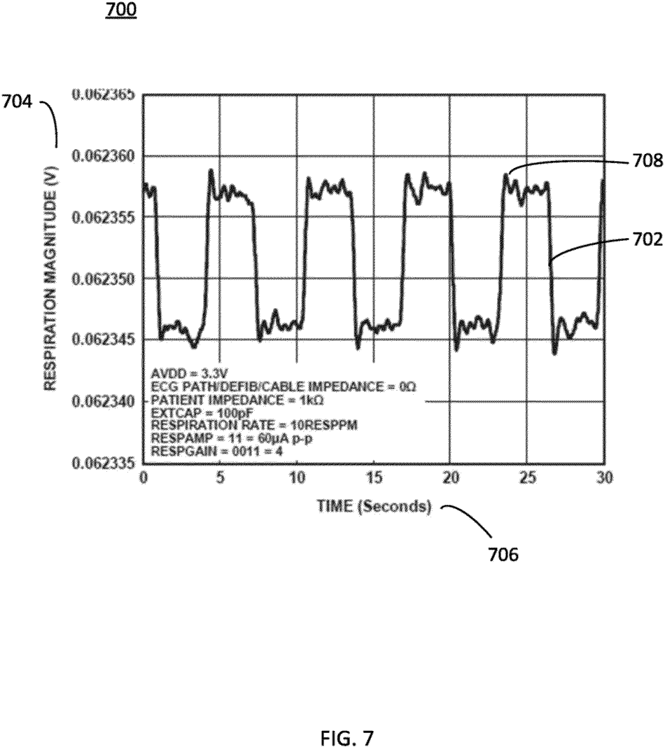

FIG. 7 shows a waveform graph 700 of an example of impedance pneumography data for patient respiration that may be generated by the wearable medical monitoring system. The waveform graph includes waveform 702 plotted with a magnitude value of respiration (v) 704 over time in seconds (s) 706. In the example, using a power supply AVDD at 3.3 V, the patient impedance is 1k Ω (kilo ohm) and impedance from the cable is 0 Ω (ohm), with a patient respiration rate of 10 resp PPM. The graph shows oscillations of impedance magnitude 708, driven by patient respiration, to determine respiration rate of the patient. In addition, a finding of an absolute magnitude of the respiration signal from the impedance pneumography data may be used to estimate the depth of respiration as a factor in determining hypopnea. Devices used to acquire ECG signals for thoracic impedance measurements include ECG (such as a 5 channel ECG) that also includes respiration detection and/or pace detection, such as an ADAS1000 ECG Analog Front End device by Analog Devices. Other pneumography techniques and devices may be employed.

A high frequency (for example, 46.5 kHz to 64 kHz) differential current may be driven into two sensing electrodes. The patient breathing causes a differential voltage from impedance variation that is measured. The modulation depth can be reduced by resistance from components, such as protection filters, a cable, and the interface between the sensing electrode and skin surface. The variation in voltage may be amplified, filtered, and synchronously demodulated. A resulting digital signal may represent the total thoracic or respiration impedance, including cable and electrode contributions.

Returning back to block 608 in FIG. 6, DC voltage measurements may also be used to acquire respiration data for analysis. DC voltage measurements may employ dry electrodes, e.g. sensing electrodes 206 in FIG. 2A and/or defibrillator electrodes 264, 266 in FIG. 2B. A change of baseline ECG data can occur as the patient respiration alters the electrode-to-skin impedance. For example, using a band type support structure around the torso of the patient or other support structure that includes a torso wrapping band, a sensor may detect movement of the band as tightening and loosening of the band occurs with the patient breath. This movement may be measured as respiration data.

In decision block 610, it is determined whether the respiration data indicate a respiratory disturbance during the sleep period, according to the above described analysis of respiration data. Certain discrepancies found by the comparison of the acquired respiration data to expected respiration data are assessed to determine whether the discrepancy meets a threshold to qualify as a respiratory disturbance. If no respiratory disturbance is indicated, in block 612, the health of the patient continues to be monitored for the duration of a monitoring period while the patient wears the monitoring system.

In block 614, in response to a finding of a respiratory disturbance during the sleep period, various sleep data are collated according to sleep factors into a sleep disorder index for the patient. The sleep disorder index that uses sleep factors with health data of the patient, is compared with a predefined sleep disorder indicator. In some implementations multiple sleep disorder indexes may be calculated using a variety of combinations of sleep factors to characterize a potential sleep disorder, by which sleep disorder events may be identified. Each index for particular sleep disorder may use sleep factors defining specific characteristics of a patient respiration, heart rate (via ECG data), and sleep, as well as other possible sleep factors, according to the acquired health data of the patient.

In some implementations, sleep factors used to determine potential sleep apnea of a patient may include the sleep data indicating times that the patient is asleep during a sleep period defined in block 606. Such sleep data that reveals when the patient is asleep may include signals acquired by sleep sensors (such as 204 in FIG. 2A), which may be a motion sensor. For example, an amount of motion of the patient during the sleep period may be used, based on a signal from the motion sensor to determine the patient is asleep.

There are various types of sleep apnea that may be determined by the monitoring system, and each type of sleep apnea may be associate with a different sleep disorder index using different or the same combinations of sleep factors. For example, obstructive sleep apnea (OSA) may be assessed using particular acquired health data indicating whether the upper air passage becomes occlusion or obstruction during sleep. The OSA condition causes the patient to stop breathing for periods of time, for example, 30 to 120 seconds in duration, and can occur many times per hour during the course of a night.

Central sleep apnea (CSA) is characterized by lack of brain function to control breathing during sleep. Cheyne-Stokes breathing is a type of central sleep apnea associated with congestive heart failure or stroke. Acquired health data may indicate whether breathing gradually increases and then decreases in breathing effort and airflow, characterizing CSA. During the weakest breathing effort, a total lack of airflow can occur.

In some implementations, the wearable medical monitoring system may determine potential levels of sleep apnea, such as mild, moderate, and severe, based on the monitoring system tracking the number of breathing failure events per a time interval, such as per hour through a night.

In some implementations, patient motion may be used as health data in certain sleep factors. For example, patient motion may be determined as a sum of the absolute values of the three axes of an accelerometer after being band pass filtered to remove any DC values. The band pass filter may be, for example, a 1 Hz high pass filter plus a 6 Hz low pass filter. Using this band pass filter as an example then, if the activity is calculated to be less than a predefined threshold value such as 0.05, then it may be considered as a lack of motion.

A threshold and criteria to use motion data to assess when a patient is asleep may be customized for typical behavior patterns of the patient. For example, the patient may routinely sleep in a raised position, such as in a raised bed or in a recliner. If the patient typically sleeps in a raised position, a body position angle (where lying flat is considered ninety degrees relative to a position perpendicular to the floor) will likely be lower than sixty degrees and higher than thirty degrees. If the angle is lower than thirty degrees, the monitoring system may determine that the patient is standing. When the typical sleeping position of the patient is detected for a period of time, such as the patient being in a raised position for ten or more minutes, a lower activity level threshold may be applied to confirm the patient is asleep. For example, if the patient is in a raised position, with a lack of motion and activity calculated as discussed above using a high pass band filter, a deflated predefined threshold value may be used to determine sleep, such as 0.025.

In some implementations torso position/orientation may be detected, such as by a 3-D accelerometer, as a sleep disorder factor for sleep apnea. Certain torso position and/or orientation may typically correlate with sleep apnea. For example, sleep apnea is less frequently found in patients who sleep in a lateral decubitus (on side) position and more common or pronounced in a supine position without elevation. Addition, sleep stage may also be used as a sleep factor in formulating a sleep disorder index for sleep apnea.

In some implementations, heath data indicating heart rate may also be used as a sleep factor in determining whether a patient is asleep. Heart rate slows down when a patient sleeps. Detection of a lower heart rate compared to when the patient entered the lying position may be used to confirm that the patient is in a deep sleep state. Further, a long term trend of heart rate may be available for the determination by the processor of the monitoring system through the memory, and may be used to more accurately confirm the sleeping state of the patient.

In some implementations, as mentioned above, the processor of the monitoring system may also use a signal from another source sensor component to confirm the patient is asleep, such as a light sensor, a clock, a respiration sensor, a sound sensor, etc. If more than one of these sensors are present, the processor may poll each of the sensors for an aggregate score and compare the score to a saved score in memory to determine the patient is asleep or awake.

A light sensor, for example, may indicate the ambience of a room since most patients sleep in darker environments. Further, a history of the ambience may be taken to help with the determination of the patient’s sleep state. A clock may also indicate the time of day and have an accumulated history indicating what time of day the patient is normally asleep. The respiration rate and pattern may also be used, as well as a sound sensor that can indicate a sleep sound of the patient, such as snoring, for example. Each of these sensors may accumulate a history to help the monitoring system accurately detect that the patient is in a sleeping state.

In some implementations, if the monitoring system determines the patient is in a lying position and the signal from the motion sensor indicates that patient has been tossing and turning more than a predefined number of times, such as 5 times, from the beginning of the lying position, then the sleep state may not be detected until the patient has been lying still for a predetermined amount of time, such as 30 minutes.

A critical sleep factor for sleep apnea may include respiration data that indicates airflow absence during the sleep period. The airflow absence may occur during a subperiod during the sleep period in which breathing pauses. Sleep factors for sleep apnea further may include a change in heart rate as indicated by the ECG data acquired by the sensing electrodes and/or defibrillator electrodes. The heart rate change may meet a threshold rate during the same subperiod in which airflow absence was detected.

A sleep disorder index may also be formulated to determine whether the patient exhibits signs of hypopnea. In some implementations, sleep factors used to determine potential hypopnea may include the sleep data indicating times that the patient is asleep during the sleep period, as described above for sleep apnea. Another sleep factor for hypopnea uses the respiration data to identify hypopnea events, which is determined to meet a hypopnea threshold level of airflow reduction. Such airflow reduction may occur during a subperiod of the defined the sleep period. In some implementations, for hypopnea the subperiod may be a sliding window that is restarted when patient motion is detected, such as by an accelerometer during the sleep period. The respiration data may include an average magnitude of respiratory complexes that is acquired during the subperiod for hypopnea.

In some implementations, the wearable medical monitoring system may determine potential levels of hypopnea, such as mild, moderate, and severe, based on the monitoring system tracking the number of hypopnea airflow reduction events per a time period, such as an hour through a night.

In assessing whether potential hypopnea exists, the wearable medical monitoring system may combine other sleep factors using various acquired health data such as oxygenation data that meets (equal to or exceeding) a hypopnea threshold level of blood oxygen desaturation in the subperiod of airflow reduction. Other health data may include the ECG data that indicates a change in heart rate corresponding with the subperiod and the respiration data.

A sleep disorder index may also be formulated to determine whether the patient exhibits signs of RERA. In some implementations, sleep factors used to determine potential RERA may include sleep data that indicates sleep period times that the patient is asleep for a portion and awake (arousal) during a portion. States of awake and asleep may be determined by acquiring certain heath data as described above for sleep apnea.

Another sleep factor for RERA evaluation applies respiration data that is determined to meet a RERA threshold level of airflow reduction during a particular subperiod of time in the sleep period. Such subperiod for RERA may be a sliding window which is restarted upon detection of patient motion, such as with an accelerometer. The respiration data may show an average magnitude of respiratory complexes acquired during this subperiod.

Evaluation for RERA may further include a negative sleep factor of detection of desaturation of oxygen level above a threshold, which may be a counterindication of RERA but rather may be a positive sleep factor for hypopnea. Thus, detection of oxygen levels in the blood may differentiate between RERA and hypopnea.

In some implementations, the wearable medical monitoring system may determine potential levels of RERA, such as mild, moderate, and severe, based on the monitoring system tracking the number of RERA airflow reduction events over a time interval, such as per hour through a night.

The sleep disorder index may be formulated for a signal type of sleep disorder or for a combination of various sleep disorders. In some implementations, a sleep apnea warning index (SAWI) may be determined using a formula to determine only a sleep apnea index may include:

$$\text{Sleep} \text{Apnea} \text{Index} = \frac{\text{(Total} \text{\#} \text{Apnea} \text{Events} \text{during} \text{a} \text{sleep} \text{subperiod)}}{\text{(Total} \text{Sleep} \text{time} \text{indentified} \text{during} \text{the} \text{sleep} \text{subperiod)}}$$

In some implementations, a formula to determine a combined sleep apnea-hypopnea index may include:

$$\text{Sleep} \text{Hypopnea-Apnea} \text{Index} = \frac{\begin{matrix}( T o t a l \# A p n e a E v e n t s d u r i n g a s l e e p s u b p e r i o d + \\ T o t a l \# H y p o p n e a E v e n t s d u r i n g t h e s l e e p p e r i o d )\end{matrix}}{\begin{matrix} \\ \overline{\text{(Total} \text{Sleep} \text{time} \text{identified} \text{during} \text{the} \text{subperiod)}}\end{matrix}}$$

In some implementations, a formula to determine a combined sleep apnea-hypopnea-RERA index may include:

$$\text{Sleep} \text{Hypopnea-Apnea-RERA} \text{Index =} \frac{\begin{matrix}( \text{Total} \text{\#} \text{Apnea} \text{Events} \text{during} \text{a} \text{sleep} \text{subperiod} \text{+} \\ \text{Total} \text{\#} \text{Hypopnea} \text{Events} \text{during} \text{the} \text{sleep} \text{period} \text{+} \\ \text{Total} \text{\#} \text{RERA} \text{Events} \text{during} \text{the} \text{sleep} \text{period)}\end{matrix}}{( \text{Total} \text{Sleep} \text{time} \text{identified} \text{during} \text{the} \text{subperiod} )}$$

The events in the above formulas represents a singular occurrence characteristic of the sleep disorder experienced by the patient. An event is determined by a collection of sleep factors as described above for sleep disorders, to which the sleep data is applied.

In some implementations, the sleep disorder index may be calculated as described above for (SAWI_ApneaOnly, SAWI_ApneaHypopnea and SAWI_ApneaHypopneaRERA), but can also be subdivided into patient torso orientation “bins” that represent the patient torso orientation during the specific event. Torso orientation may be determined with use of a sensor such as a 3-axis accelerometer attached to the support structure in a known orientation relative to the patient’s torso. Patient torso orientations that may be used to subdivide data include, but are not limited to:
[230] (a) Patient Supine (laying on their back, such as at 0 degree)
[231] (b) Patient Prone (laying on their stomach, such as at 0 degrees)
[232] (c) Patient Torso Lateral Right (laying on their right side)
[233] (d) Patient Torso Lateral Left (laying on their left side)
[234] (e) Patient Torso Propped Up (face forward with torso at an angle less than (<) 90 degrees)

Other sleep disorders may be identified according to the processes described in FIG. 6 with various combinations of sleep factors that point to the particular sleep disorders.

In decision block 616 of the flowchart in FIG. 6, the comparison of a sleep disorder index with a sleep disorder indicator in block 614 described above, is used to determine a potential sleep disorder for the patient. For example, where the sleep disorder index for the patient maps, within an allowable variation amount, to the sleep disorder indicator for a particular sleep disorder, it is determined that the particular sleep disorder may be present for the patient.

In some implementations, a neural network may be employed to determine a sleep disorder index for particular sleep disorders and in some implementations may also determine whether a sleep disorder is likely presented by the patient. For example, the neural network may receive as input various sleep data. The neural network processes the data to output a sleep disorder index for the patient and/or output a prediction of a particular sleep disorder for the patient. The neural network may be trained with health data of various combinations of sleep factors that together indicate a high probability of the particular sleep disorder. Feedback data may be attained by confirmation of a sleep disorder by additional testing, such as during a sleep study, of a patient whose health data is used by the neural network in formulating a sleep disorder index. Such feedback data may be used as training input data to retrain the neural network.

In block 616, the monitoring of the patient health continues until an end time of the monitoring session, e.g., a time that the wearable monitoring system is no longer being used by the patient, or a time that sleep disorder monitoring is discontinued, for example in cases that a health support entity is satisfied that no sleep disorder exists. In some implementations, the respiration data and other related information may be transmitted to a computing device of a health support entity for status information of the patient.

If a potential sleep disorder is found, in block 618, the sleep disorder information is transmitted to the health support entity, for example, in the form of a warning of the sleep disorder.

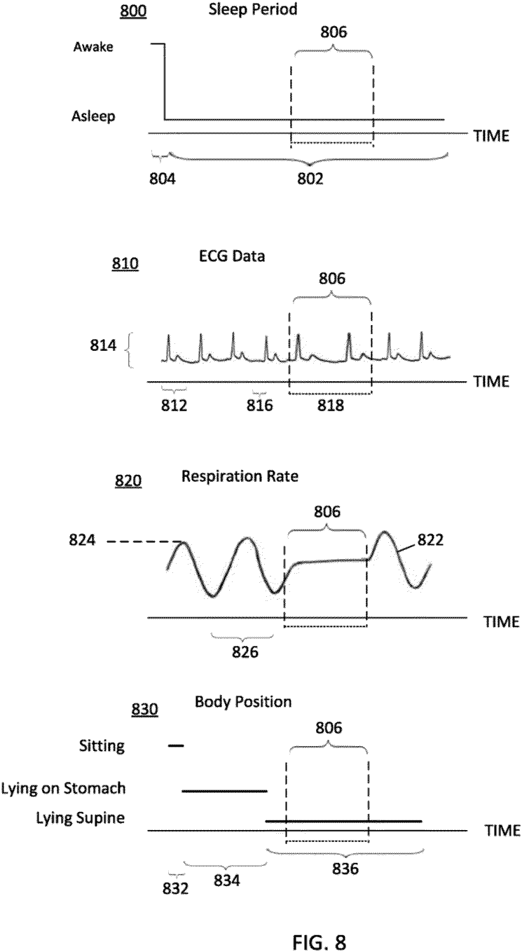

FIG. 8 shows are example graphs and waveforms for various health data during a sleep period indicating potential sleep apnea of a patient, in accordance with some implementations. A sleep state data graph 800 shows a sleep period 802 and an awake period 804. For example, the sleep state graph may indicate an awake period just before the patient goes to bed or where the patient was awakened at night and subsequently goes back to sleep. In some implementations, the sleep period may be represented by an interval during a time of day, such as 9:00 PM to 6:00 AM, and corresponding date. The sleep period graph 800 may represent a sample or portion of a sleep period that may encompass the course of a night. The sleep period 802 shown may include a subperiod of time 806 that may be flagged based on variations in other acquired health data described below, which corresponds with the subperiod.

An ECG data waveform graph 810 shows ECG data from ECG signals that correlate with the sleep period 802 shown in the sleep graph 800. The ECG data includes QRS complexes 812 from which variations in a baseline of ECG signal may be identified. Each QRS 812 has a peak or magnitude 814. In a regular cycle, peaks occur regularly within particular time phrases with a duration gap 816 between ECG waves. A line segment 818 shows an instance during subperiod 806, where it is detected that the duration gap defined between the two identified QRS complexes greatly increases and less peaks (QRS complexes) are generated, representing a slowdown of heart rate.

A respiratory rate waveform graph 820 shows over the sleep period 802 each wave 822 has a magnitude 824 and occurs at regular intervals 826. It is detected that during subperiod 806, the duration gap greatly increases to during which no respiration wave peaks are generated. Lack of wave peaks represents absence of breathing during the subperiod 806.

A detected body position graph 830 shows various patient positions during the awake prior 804 and sleep period 802 of the sleep graph 800. During the awake period 804, the patient is found to be in a sitting position. Although, a patient may be asleep while sitting, it is not generally a position that suggests the patient is sleeping. While in the sitting position, a determination of sleep may be found using a combination of sleep period factors.

During a first portion 832 of the sleep period 802, the patient is sensed to be sitting. During a second portion 834 of the sleep period 802, the patient is sensed to be lying on the stomach. During a third portion of the sleep period 836, the patient is detected to be lying supine, which includes the subperiod 806. The body position during subperiod 806 is used a sleep factor for sleep apnea. By the monitoring system collating the health data from graphs 800, 810, 820, 830 the monitoring system flags the patient for sleep apnea based on deviations in the ECG data, variations in the respiration data, and sleep position occurring during the same subperiod 806. A warning of possible sleep apnea may be transmitted to a health support entity.

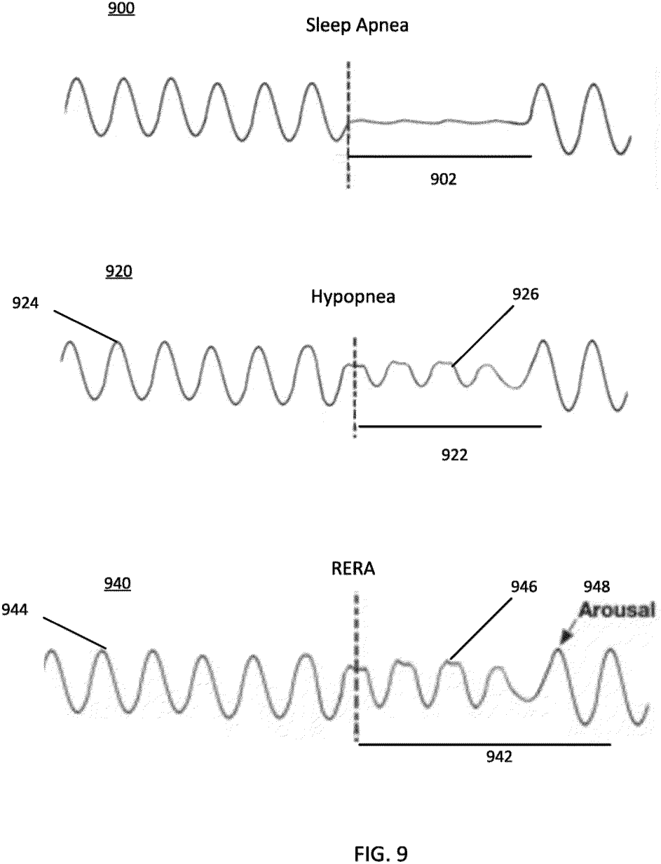

FIG. 9 are example respiratory waveform graphs of respiratory heath data representing the course of a sleep period of time. The respiratory data may suggest sleep apnea in graph 900, hypopnea in graph 920, and RERA in graph 940. The respective respiratory pattern during the subperiods of each waveform graphs 900, 920, and 940 are applied to sleep factors used to assess the respective sleep disorders.

Sleep apnea graph 900 shows a regular breathing pattern prior to a subperiod 902, at which time breathing temporarily ceases in the patient. Sleep apnea may be characterized, at least in part, by no airflow for a defined subperiod of time, such as at least ten seconds, while sleeping.

In some implementations, sleep apnea can be estimated solely by analyzing the respiration rate health data over a predefined subperiod time during a determined sleep period and a finding of no discernable waveform over that subperiod. In other implementations, for sleep apnea determination the lack of respiration is combined with other sleep factures such as rapid change in heart rate, up or down. In still other implementations, sleep apnea can be estimated by analyzing the data for respiration rate over time, during periods when the patient is determined to be asleep, looking for subperiods of time, such as 10 seconds, with no discernable respiration waveform and further sleep factors of one or more of the following aspects: (i) a corresponding, rapid change in heart rate (up or down); (ii) a reduction in blood oxygen level as determined by a separate SpO2 sensor whose data can be time-synchronized to the ECG and respiration data.

Hypopnea graph 920 shows regular a breathing pattern prior to the beginning of a subperiod 922, at which time the breathing continues at a small percentage of the regular breathing pattern. For example, wave magnitude at 924 prior to the subperiod 922 changes to a wave magnitude 926 during the subperiod 922. Hypopnea may be characterized, at least in part, by shallow breaths for a predefined period of time, such as 10 seconds or longer while asleep. For example, greater than or equal to (≥) 30% reduction in airflow with 4% blood oxygen desaturation may satisfy sleep factors for hypopnea. In other examples, greater than or equal to (≥) 50% reduction in airflow with 3% blood oxygen desaturation may satisfy sleep factors for a determination of hypopnea.

In some implementations, hypopnea may be determined by the monitoring system performing calculation of an average peak-to-peak magnitude of respiration complexes over a sliding window of a time period, referred to as RespComplexMagnitude_{AVG}. Respiration complexes are identified whose peak-to-peak amplitude, relative to RespComplexMagnitude_{AVG}, is less than a programmable HypopneaPercentThreshold value (e.g., 40%). Identification is made of subperiods (time segments) which are of a predetermined length of time, such as greater than or equal to (≥) 10 seconds in length, in which the relative magnitude of all respiration complexes during the subperiod fail to meet (are less than) the HypopneaPercentThreshold.

In other implementations, hypopnea can be estimated by a 3-axis accelerometer method and analyzing the respiration rate health data over a time along with acquired patient motion data detected by the 3-axis accelerometer attached to the support structure of the monitoring system in a known orientation relative to the patient’s torso. Calculations according to this process of identifying hypopnea are performed by the monitoring system to determine an average peak-to-peak magnitude of respiration complexes over a sliding window time period, referred to as RespComplexMagnitude_{AVG}. Respiration complexes are identified whose peak-to-peak amplitude, relative to RespComplexMagnitude_{AVG}, is less than a programmable HypopneaPercentThreshold value (e.g., 40%). If patient motion is detected on the 3-axis accelerometer data e.g., torso rotation or torso upright angle, it is likely that respiration magnitudes have changed. In response to detecting patient motion, a sliding-window calculation is performed of average respiration magnitudes. Identification of smaller respiration complexes may be suspended until a defined number of respiration complexes have been averaged with no altering patient motion. Period(s) of time is/are identified, such as greater than or equal to (≥) 10 seconds in length, where the relative magnitude of all respiration complexes during the period are less than HypopneaPercentThreshold.

In performing the hypopnea assessment, additional combinations of sleep factors may be employed. For example, the above described hypopnea assessments with or without 3-axis accelerometer data, may also be combined with a sleep factor of rapid change in heart rate up or down. Another combination of sleep factors for hypopnea may include (i) a corresponding, rapid change in heart rate (up or down); and (ii) a reduction in blood oxygen level as determined by a separate SpO2 sensor whose data can be time-synchronized to the ECG and respiration data. Other combinations of sleep factors may be used in hypopnea assessments as are recognized by those well-skilled in the art after careful review of the present disclosure.

The RERA waveform graph 940 shows regular breathing patter prior to a subperiod 942, which time the breathing patter changes to a smaller intensity than the regular breathing pattern during a first portion of the subperiod. The breathing intensity is represented by a smaller magnitude peak 946 in the subperiod 942 than the reduced magnitude peak 926 in the hypopnea graph 920 during the subperiod 922. An arousal moment 948 is also detected in the patient at a second end portion of the subperiod 942 occurring after the first portion. Thus, the subperiod 942 may be divided into portions having different breathing patterns. The arousal moment 948 is a differentiating feature the RERA graph 940 from the hypopnea graph 920.

In some examples, as a small reduction in airflow for a predefined period of time, such as 10 seconds or more while sleeping as well as sleep factors for sleep apnea and hypopnea not being met may satisfy sleep factors for RERA.

RERA can be estimated by analyzing the data for respiration rate over time along with acquired patient motion data detected by a 3-axis accelerometer attached to the support structure of the monitoring system in a known orientation relative to the torso of the patient. The wearable medical monitoring system may calculate an average peak-to-peak magnitude of respiration complexes over a sliding window time period, referred to as RespComplexMagnitude_{AVG}. The monitoring system may identify respiration complexes whose peak-to-peak amplitude, relative to RespComplexMagnitude_{AVG}, is greater than a programmable HypopneaPercentThreshold value (e.g., 40%) but less than a programmable RERAPercentThreshold (e.g., 70%) value. If patient motion is detected on the 3-axis accelerometer data, e.g., torso rotation or torso upright angle, it may be assumed that respiration magnitudes have changed.

In response to detecting patient motion, the calculations may be restarted in a sliding-window to determine average respiration magnitudes. In addition, identification of smaller respiration complexes may be suspended until a defined number of respiration complexes have been averaged with no altering patient motion.

Periods of defined lengths of time, e.g. greater than or equal to (≥) 10 seconds in length, may be identified by the monitoring system as potentially RERA, in which the criteria for hypopnea, as described above using a 3-axis accelerometer, are not met but the relative magnitude of all respiration complexes during the period are less than a RERAPercentThreshold. The identified period of time may be classified as respective RERA events if patient motion is detected during the interval of time when the relative magnitude of all respiration complexes during the period are less than RERAPercentThreshold.

The devices and/or systems described in this document perform functions, processes and/or methods. These functions, processes and/or methods may be implemented by one or more devices that include logic circuitry. Such a device can be alternately called a computer, and so on. It may be a standalone device or computer, such as a general purpose computer, or part of a device that has one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described above. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description includes flowcharts, display images, algorithms, and symbolic representations of program operations that may be provided within at least one non-transitory, tangible, computer readable medium for execution by the one or more processors. An economy is achieved in that a flowchart as in FIG. 6 is used to describe both programs, and also methods. So, while flowcharts described methods in terms of boxes, they also concurrently describe programs.

We claim:

1. A medical system for health monitoring of a patient, comprising:
a support structure configured for long term wear on the patient, the support structure including:
electrodes positioned to detect electrical signals to generate electrocardiogram (ECG) data; and
one or more sleep sensors to produce sleep signals;
a respiration detector to generate respiration data;
one or more sleep detectors to generate sleep data from the sleep signals; and
at least one processor configured to use logic to perform operations comprising:
determining a cardiac condition based, at least in part, on the ECG data;
determining at least one respiratory disturbance during a sleep period based, at least in part, on the respiration data; and
determining a potential sleep disorder by comparing a sleep disorder index based on sleep factors, with a sleep disorder indicator, wherein the sleep factors are associate with two or more health data selected from a group consisting of: the respiration data, the ECG data, and the sleep data.

2. The system of claim 1, where in the support structure is configured for long term wear on a torso of the patient.

3. The system of claim 1, further comprising a communication component, wherein the operations of the at least one processor includes:
causing the communication component to transmit a warning of the potential sleep disorder to a patient support device.

4. The system of claim 1, wherein the potential sleep disorder includes sleep apnea, and wherein the sleep factors include:
an indication of time that the patient is asleep during the sleep period according to the sleep data;
an indication that a first subperiod of airflow is absent during the sleep period according to the respiration data; and
an indication of a change in heart rate at a threshold rate corresponding with the first subperiod of airflow absence according to the ECG data.

5. The system of claim 4, wherein the sleep data includes a torso position of the patient during the sleep period, and
wherein the sleep factors includes an indication of a flat or angled supine position of the patient according to the sleep data.

6. The system of claim 1, wherein the potential sleep disorder includes hypopnea, wherein the sleep factors include:
an indication of a time that the patient is asleep during a sleep period according to the sleep data;
the respiration data meeting a hypopnea threshold level of airflow reduction in a second subperiod during the sleep period; and
one or more additional sleep factors selected from a group of: oxygenation data meeting a hypopnea threshold level of blood oxygen desaturation in the second subperiod, and an indication of a change in heart rate corresponding with the respiration data according to the ECG data.

7. The system of claim 6, wherein the second subperiod is a sliding window that is restarted upon a detection of patient motion during the sleep period, and wherein the respiration data includes an average magnitude of respiratory complexes acquired during the second subperiod.

8. The system of claim 7, wherein the one or more sleep sensors comprise an accelerometer that detects the patient motion.

9. The system of claim 1, wherein the potential sleep disorder includes respiratory effort related arousal (RERA), and wherein the sleep factors include:
an indication of a sleep period in which the patient is asleep during a first portion of the sleep period and awake during a second portion of the sleep period according to the sleep data; and
the respiration data meeting a RERA threshold level of airflow reduction during a third subperiod of time during the sleep period.

10. The system of claim 9, wherein the third subperiod is a sliding window and the respiration data includes an average magnitude of respiratory complexes acquired during the third subperiod, wherein the third subperiod is restarted upon a detection of patient motion.

11. The system of claim 1, wherein the one or more sleep sensors comprise an accelerometer and the sleep data detected by the accelerometer includes patient motion data and/or torso position data.

12. The system of claim 1, wherein the sleep data comprises time of day data obtained by a clock and used to determine if a time of day is within a predefined sleep period and a predefined awake period.

13. The system of claim 1, wherein the respiration detector receives respiratory impedance signals from alternating current (AC) signals at the electrodes or receives direct current (DC) signals at the electrodes.

14. A method to monitor health of a patient with a wearable article, the method comprising:
providing a support structure configured for long term wear on the patient, wherein the support structure includes electrodes positioned to detect electrical signals to generate electrocardiogram (ECG) data; and one or more sleep sensors to produce sleep signals;
providing a respiration detector to generate respiration data; and
providing a sleep detector to acquire sleep data from the sleep signals;
determining a cardiac condition based, at least in part, on the ECG data;
determining at least one respiratory disturbance during a sleep period based, at least in part, on the respiration data; and
determining a potential sleep disorder by comparing a sleep disorder index based on sleep factors, with a sleep disorder indicator, wherein the sleep factors are associate with two or more health data selected from a group consisting of: the respiration data, the ECG data, and the sleep data.

15. The method of claim 14, where in the support structure is configured for long term wear on a torso of the patient.

16. The method of claim 14, further comprising:
causing a communication component to transmit a warning of the potential sleep disorder to a communication device of a patient support user.

17. The method of claim 14, wherein the potential sleep disorder includes sleep apnea, and wherein the sleep factors include:
an indication of a time that the patient is asleep during a sleep period according to the sleep data;
an indication of a first subperiod of airflow absence during the sleep period according to the respiration data; and
an indication of a change in heart rate at a threshold rate corresponding with the first subperiod of airflow absence according to the ECG data.

18. The method of claim 14, wherein the potential sleep disorder includes hypopnea, wherein the sleep factors include:
an indication of a time that the patient is asleep during a sleep period according to the sleep data;
the respiration data meeting a hypopnea threshold level of airflow reduction in a second subperiod during the sleep period; and
one or more additional sleep factors selected from a group of: oxygenation data meeting a hypopnea threshold level of blood oxygen desaturation in the second subperiod, an indication of a change in heart rate corresponding with the respiration data according to the ECG data, and combinations thereof.

19. The method of claim 14, wherein the potential sleep disorder includes RERA, and wherein the sleep factors include:
an indication of a sleep period in which the patient is asleep during a first portion of the sleep period and awake during a second portion of the sleep period according to the sleep data; and
the respiration data meeting a RERA threshold level of airflow reduction during a third subperiod of time during the sleep period.

20. The method of claim 14, wherein the one or more sleep sensors include an accelerometer and the sleep data includes patient motion data and/or torso orientation data.
